SEQUENCE LISTING (44 sequences)

SEQ ID NO: 1 - DNA, 67 bp, Artificial Sequence
  Synthetic polynucleotide
ctgttggtggtgcaggaggcattgccttctaccctccagatggtcggttataaattctgattagcca

SEQ ID NO: 2 - DNA, 73 bp, Artificial Sequence
  Synthetic polynucleotide
ctgttggtggtgcaggaggcattgcagagactaccgctgatccagatggtcggttataaattctgattagcca

SEQ ID NO: 3 - DNA, 135 bp, Artificial Sequence
  Synthetic polynucleotide
catcccgctcaatcacgacagcgagatccctccaaaatcaagtggaactcaactttaccttcatcctggtaatccaggatgtgcgcttctaccctcatggagggtagaaggcaatgcctcctgcaccaccaacag

SEQ ID NO: 4 - DNA, 26 bp, Artificial Sequence
  Synthetic polynucleotide
tggctaatcagaatttataaccgacc

SEQ ID NO: 5 - DNA, 20 bp, Artificial Sequence
  Synthetic polynucleotide
catcccgctcaatcacgaca

SEQ ID NO: 6 - DNA, 18 bp, Artificial Sequence
  Synthetic polynucleotide
tgttggtggtgcaggagg

SEQ ID NO: 7 - DNA, 67 bp, Artificial Sequence; identical to SEQ ID NO: 1
  Synthetic polynucleotide

SEQ ID NO: 8 - DNA, 80 bp, Artificial Sequence
  Synthetic polynucleotide
tggctaatcagaatttataaccgaccatctggagggtagaagcgcacatcctggattaccaggatgaaggtaaagttgag

SEQ ID NO: 9 - DNA, 135 bp, Artificial Sequence
  Synthetic polynucleotide
tcctccagcttcttctgcaggcgagatccctccaaaatcaagtggaactcaactttaccttcatcctggtaatccaggatgtgcgcttctaccctcatggagggtagaaggcaatgcctcctgcaccaccaacag

SEQ ID NO: 10 - DNA, 80 bp, Artificial Sequence
  Synthetic polynucleotide
ctcaactttaccttcatcctggtaatccaggatgtgcgcttctaccctccagatggtcggttataaattctgattagcca

SEQ ID NO: 11 - DNA, 26 bp, Artificial Sequence
  Synthetic polynucleotide
tggctaatcagaatttataaccgacc

SEQ ID NO: 12 - DNA, 20 bp, Artificial Sequence
  Synthetic polynucleotide
tcctccagcttcttctgcag

SEQ ID NO: 13 - DNA, 121 bp, Artificial Sequence
  Synthetic polynucleotide - Stopper-3-3-0
tcctccagcttcttctgcaggcgagatccctccaaaatcaagtggaactcaactttaccttcatcctggtaatccaggatgtgcgtatagccactggcaatgcctcctgcaccaccaacag

SEQ ID NO: 14 - DNA, 129 bp, Artificial Sequence
  Synthetic polynucleotide - Stopper-4-4-0
tcctccagcttcttctgcaggcgagatccctccaaaatcaagtggaactcaactttaccttcatcctggtaatccaggatgtgcgtggcctacagcagtagaaggcaatgcctcctgcaccaccaacag

SEQ ID NO: 15 - DNA, 129 bp, Artificial Sequence
  Synthetic polynucleotide - Stopper-4-4-2
tcctccagcttcttctgcaggcgagatccctccaaaatcaagtggaactcaactttaccttcatcctggtaatccaggatgtgcgtggcctacatgggtagaaggcaatgcctcctgcaccaccaacag

SEQ ID NO: 16 - DNA, 129 bp, Artificial Sequence
  Synthetic polynucleotide - Stopper-4-4-4
tcctccagcttcttctgcaggcgagatccctccaaaatcaagtggaactcaactttaccttcatcctggtaatccaggatgtgcgtggcctactagggtagaaggcaatgcctcctgcaccaccaacag

SEQ ID NO: 17 - DNA, 156 bp, Artificial Sequence
  Synthetic polynucleotide - Stopper-4-8-5
tcctccagcttcttctgcaggcgagatccctccaaaatcaagtggaactcaactttaccttcatcctggtaatccaggatgtgcgtggccaccaccatggagaaggctgggcaatgcctcctgcaccaccaacagcccgccggaaacttcttccca

SEQ ID NO: 18 - DNA, 135 bp, Artificial Sequence
  Synthetic polynucleotide - Stopper-8-4-2
tcctccagcttcttctgcaggcgagatccctccaaaatcaagtggaactcaactttaccttcatcctggtaatccaggatgtgcgtggcccttctaccggagggtagaaggcaatgcctcctgcaccaccaacag

SEQ ID NO: 19 - DNA, 135 bp, Artificial Sequence
  Synthetic polynucleotide - Stopper-8-5-2
tcctccagcttcttctgcaggcgagatccctccaaaatcaagtggaactcaactttaccttcatcctggtaatccaggatgtgcgtggccttctacctggagggtagaaggcaatgcctcctgcaccaccaacag

SEQ ID NO: 20 - DNA, 160 bp, Artificial Sequence
  Synthetic polynucleotide - Stopper-8-30-0
tcctccagcttcttctgcaggcgagatccctccaaaatcaagtggaactcaactttaccttcatcctggtaatccaggatgtgcgtggccttctaccaagtgtgtgaaccgcaagttctgtacaagcggtagaaggcaatgcctcctgcaccaccaacag

SEQ ID NO: 21 - DNA, 160 bp, Artificial Sequence
  Synthetic polynucleotide - Stopper-8-30-20
tcctccagcttcttctgcaggcgagatccctccaaaatcaagtggaactcaactttaccttcatcctggtaatccaggatgtgcgtggccttctaccaagtgtgtgataaagtccagctccagatagggtagaaggcaatgcctcctgcaccaccaacag

SEQ ID NO: 22 - DNA, 164 bp, Artificial Sequence
  Synthetic polynucleotide - Stopper-8-34-0
tcctccagcttcttctgcaggcgagatccctccaaaatcaagtggaactcaactttaccttcatcctggtaatccaggatgtgcgtggccttctaccaagtgtgggtggaaaccgcaagttctgtacaagcggtagaaggcaatgcctcctgcaccaccaacag

SEQ ID NO: 23 - DNA, 135 bp, Artificial Sequence
  Synthetic polynucleotide - Stopper-10-4-2
tcctccagcttcttctgcaggcgagatccctccaaaatcaagtggaactcaactttaccttcatcctggtaatccaggatgtgcgtcttctaccctatggagggtagaaggcaatgcctcctgcaccaccaacag

SEQ ID NO: 24 - DNA, 135 bp, Artificial Sequence; identical to SEQ ID NO: 9
  Synthetic polynucleotide - Stopper-10-5-2

SEQ ID NO: 25 - DNA, 135 bp, Artificial Sequence
  Synthetic polynucleotide - Stopper-12-4-3
tcctccagcttcttctgcaggcgagatccctccaaaatcaagtggaactcaactttaccttcatcctggtaatccaggatgtcttctaccctatccatatagggtagaaggcaatgcctcctgcaccaccaacag

SEQ ID NO: 26 - DNA, 135 bp, Artificial Sequence
  Synthetic polynucleotide - Stopper-12-5-3
tcctccagcttcttctgcaggcgagatccctccaaaatcaagtggaactcaactttaccttcatcctggtaatccaggatgcttctaccctataccatatagggtagaaggcaatgcctcctgcaccaccaacag

SEQ ID NO: 27 - DNA, 168 bp, Artificial Sequence
  Synthetic polynucleotide - Stopper-12-30-0
tcctccagcttcttctgcaggcgagatccctccaaaatcaagtggaactcaactttaccttcatcctggtaatccaggatgtgcgtggccttctaccctatagtgtggtgataaccgcaagttctgtaagcatagggtagaaggcaatgcctcctgcaccaccaacag

SEQ ID NO: 28 - DNA, 159 bp, Artificial Sequence
  Synthetic polynucleotide - Stopper-20-5-0
tcctccagcttcttctgcaggcgagatccctccaaaatcaagtggaactcaactttaccttcatcctggtaatccaggatgtgcgtggccttctaccctatctggagctcaagtagctccagatagggtagaaggcaatgcctcctgcaccaccaacag

SEQ ID NO: 29 - DNA, 70 bp, Artificial Sequence
  Synthetic polynucleotide
ctttaccttcatcctggtaatccaggatgtgcgcttctaccctatctggacaagtggtggaaaccgcatt

SEQ ID NO: 30 - DNA, 68 bp, Artificial Sequence
  Synthetic polynucleotide
ctttaccttcatcctggtaatccaggatgtgcgcttctaccctatctgggacgttactgacgtggtgc

SEQ ID NO: 31 - DNA, 75 bp, Artificial Sequence
  Synthetic polynucleotide
ctttaccttcatcctggtaatccaggatgtgcgcttctaccctccagatggtcggttataaattctgattagcca

SEQ ID NO: 32 - DNA, 62 bp, Artificial Sequence
  Synthetic polynucleotide
ctgttggtggtgcaggaggcattgccttctaccctatctggacaagtggtggaaaccgcatt

SEQ ID NO: 33 - DNA, 60 bp, Artificial Sequence
  Synthetic polynucleotide
ctgttggtggtgcaggaggcattgccttctaccctatctgggacgttactgacgtggtgc

SEQ ID NO: 34 - DNA, 67 bp, Artificial Sequence; identical to SEQ ID NO: 1
  Synthetic polynucleotide

SEQ ID NO: 35 - DNA, 20 bp, Artificial Sequence
  Synthetic polynucleotide
aatgcggtttccaccacttg

SEQ ID NO: 36 - DNA, 19 bp, Artificial Sequence
  Synthetic polynucleotide
gcaccacgtcagtaacgtc

SEQ ID NO: 37 - DNA, 26 bp, Artificial Sequence
  Synthetic polynucleotide
tggctaatcagaatttataaccgacc

SEQ ID NO: 38 - DNA, 135 bp, Artificial Sequence; identical to SEQ ID NO: 9
  Synthetic polynucleotide

SEQ ID NO: 39 - RNA, 61 nt, Artificial Sequence
  Synthetic polynucleotide
cuguugguggugcaggaggcauugccuucuacccuaucuggaguacgaacgccaucgacuu

SEQ ID NO: 40 - RNA, 120 nt, Artificial Sequence
  Synthetic polynucleotide
caggcgagaucccuccaaaaucaaguggaacucaacuuuaccuucauccugguaauccaggaugugcguggccaccaccauggagaaggcugggcaaugccuccugcaccaccaacagcc

SEQ ID NO: 41 - DNA, 20 bp, Artificial Sequence
  Synthetic polynucleotide
aagtcgatggcgttcgtact

SEQ ID NO: 42 - DNA, 18 bp, Artificial Sequence
  Synthetic polynucleotide
caggcgagatccctccaa

SEQ ID NO: 43 - DNA, 63 bp, Artificial Sequence
  Synthetic polynucleotide
ggatgtgcgtggccaccaccatggagaaggcattgccttctaccctatctggagaacgaacgc

SEQ ID NO: 44 - DNA, 72 bp, Artificial Sequence
  Synthetic polynucleotide
aagtcgatggcgttcgtactccagatagggtagaaggcaatgccttctccatggtggtggccacgcacatcc

(19) United States
(12) Patent Application Publication
(10) Pub. No.: US 2024/0218435 A1
(43) Pub. Date: Jul. 4, 2024

(54) COMPOSITIONS AND METHODS FOR CHIMERIC AMPLICON FORMATION

(71) Applicant: William Marsh Rice University, Houston, TX (US)

(72) Inventors: David ZHANG, Houston, TX (US); Kerou ZHANG, Houston, TX (US); Ping SONG, Houston, TX (US)

(73) Assignee: William Marsh Rice University, Houston, TX (US)

(21) Appl. No.: 18/557,747

(22) PCT Filed: Apr. 29, 2022
(86) PCT No.: PCT/US2022/026980

§ 371 Date: Oct. 27, 2023

Related U.S. Application Data
(60) Provisional application No. 63/182,154, filed on Apr. 30, 2021.

(51) Int. Cl. C12Q 1/6844

(52) U.S. Cl. CPC C12Q 1/6844

(57) ABSTRACT

Provided herein are compositions and methods for formation of amplicons having a chimeric sequence, partially derived from a target nucleic acid and partially derived from a rationally designed oligonucleotide. The provided compositions provide for high-yield, induced template switching between the target nucleic acid and the rationally designed oligonucleotide as the template during polymerase extension, achieving inheritance of information from both within only one cycle.

### REFERENCE TO RELATED APPLICATIONS

The present application claims the priority benefit of U.S. provisional application No. 63/182,154, filed Apr. 30, 2021, the entire contents of which is incorporated herein by reference.

### STATEMENT REGARDING FEDERALLY SPONSORED RESEARCH

This invention was made with government support under Grant No. R01CA203964 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Apr. 25, 2022, is named RICEP0084WO_ST25.txt and is 13,303 bytes in size.

### BACKGROUND

### 1. Field

The present invention relates generally to the field of molecular biology. More particularly, it concerns compositions and methods for formation of amplicons with a chimeric sequence inherited from two different nucleic acid molecules.

### 2. Description of Related Art

PCR and ligation are frequently used methods in sequencing library preparation to append adapter sequences to both the 5′ and 3′ ends of nucleic sequences of interest. Both of the methods have some limitations. The efficiency of ligation is usually low, at between 10% and 30%, failing to add adapter sequences to the majority of molecules, leading to the loss of those molecules. The PCR method requires at least two cycles to append the adapter sequence to both ends of the amplicon molecules. This means that either a thermostable polymerase must be used, or that additional polymerase must be added to the reaction after the first cycle. Methods are needed to overcome the limitations of thermostable polymerases and thermo-cycling reactions as well as low efficiency of ligation.

### SUMMARY

In one embodiment, provided herein are compositions comprising: (a) a Primer oligonucleotide, and (b) a Stopper oligonucleotide, wherein the Stopper comprises from 5′ to 3′: (i) a First Sequence with a length between 5nt and 200nt, (ii) a Second Sequence with a length between 3nt and 50nt, (iii) a Loop Sequence with a length between 3nt and 70nt, (iv) a Third Sequence with a length between 3nt and 50nt, wherein the Third Sequence is complementary to the Second Sequence, and (v) a Fourth Sequence with a length between 6nt and 500nt, wherein the Fourth Sequence is complementary to a Binding Region sequence on a Target nucleic acid, wherein the Third Sequence is complementary to a Match Region sequence positioned to the 3′ of the Binding Region on the Target nucleic acid, and wherein a 3′ subsequence of the Primer comprising at least 15 nucleotides is (at least 80%) complementary to a Priming Region sequence positioned to the 3′ of the Match Region on the Target nucleic acid. The complementarity relationships described above may be less than 100% complementarity. In some aspects, the complementarity relationships described above are ≥95%, ≥90%, ≥85%, or ≥80% complementarity. In some aspects, the composition is for forming a chimeric amplicon of a Target nucleic acid by polymerase extension, wherein the Target nucleic acid comprises, from 5′ to 3′, a Binding Region, a Match Region, and a Priming Region. The composition may be used for formation of amplicons with chimeric sequence inherited from both a Target nucleic acid molecule and a Stopper nucleic acid molecule. The composition may be used for inducing template switching by polymerases. The composition may be used for preparing sequencing libraries.

In one embodiment, provided herein are compositions for forming a chimeric amplicon of a Target nucleic acid by polymerase extension, wherein the Target nucleic acid comprises, from 5′ to 3′, a Binding Region, a Match Region, and a Priming Region, the composition comprising a Stopper oligonucleotide, wherein the Stopper comprises from 5′ to 3′: (a) a First Sequence with a length between 5nt and 200nt, (b) a Second Sequence with a length between 3nt and 50nt, (c) a Loop Sequence with a length between 3nt and 70nt, (d) a Third Sequence with a length between 3nt and 50nt, wherein the Third Sequence is complementary to the Second Sequence, and (e) a Fourth Sequence with a length between 6nt and 500nt, wherein the Fourth Sequence is complementary to the Binding Region of the Target nucleic acid, and wherein the Third Sequence is complementary to the Match Region of the Target nucleic acid. In some aspects, the composition further comprises a Primer oligonucleotide, wherein a 3′ subsequence of the Primer comprising at least 15 nucleotides is complementary to a Priming Region sequence positioned to the 3′ of the Match Region on the Target nucleic acid. The complementarity relationships described above may be less than 100% complementarity. In some aspects, the complementarity relationships described above are ≥95%, ≥90%, ≥85%, or ≥80% complementarity. The composition may be used for formation of amplicons with chimeric sequence inherited from both a Target nucleic acid molecule and a Stopper nucleic acid molecule. The composition may be used for inducing template switching by polymerases. The composition may be used for preparing sequencing libraries.

In some aspects, the composition further comprises the Target nucleic acid. In some aspects, the Match Region is positioned immediately to the 3′ of the Binding Region. In some aspects, the Match Region is adjacent to the Binding Region.

In some aspects, the composition further comprises a template-dependent polymerase enzyme. In some aspects, the template-dependent polymerase enzyme is thermostable. In some aspects, the template-dependent polymerase enzyme is not thermostable.

In some aspects, the composition further comprises reagents and buffers needed for polymerase function.

In some aspects, the Primer comprises a 5′ subsequence that is not complementary to a region of the Target nucleic acid positioned 3′ of the Priming Region. In some aspects, the Primer comprises a 5′ subsequence that is not complementary to a region of the Target nucleic acid positioned immediately 3′ of the Priming Region. In some aspects, the Primer comprises a 5′ subsequence that is not complementary to a region of the Target nucleic acid positioned within a 20-nucleotide region 3′ of the Priming Region. In some aspects, the Primer comprises a 5′ subsequence that comprises a sequencing adaptor or index sequence.

In some aspects, the Stopper oligonucleotide further comprises a Fifth Sequence between the Second Sequence and the Loop Sequence, and a Sixth Sequence between the Loop Sequence and the Third Sequence, wherein the Fifth Sequence is complementary to the Sixth Sequence. In some aspects, the Sixth Sequence is not complementary to a region of the Target nucleic acid positioned 3′ of the Match Region. In some aspects, the Fifth Sequence is not complementary to a region of the Target nucleic acid positioned immediately 3′ of the Match Region. In some aspects, the Fifth Sequence is not complementary to a region of the Target nucleic acid positioned within a 20-nucleotide region 3′ of the Match Region

In some aspects, the Stopper oligonucleotide has a subsequence at the 3′ end at least 3 nucleotides long that is not complementary to the Target. In some aspects, the subsequence at the 3′ end forms at least one hairpin structure. In some aspects, the Stopper oligonucleotide comprises non-natural nucleotides. In some aspects, the Stopper oligonucleotide has a chemical functionalization at the 3′ end that prevents polymerase extension. In some aspects, the chemical functionalization is selected from the group consisting of a 3-carbon spacer, an inverted nucleotide, and a minor groove binder.

In some aspects, the Primer oligonucleotide is a DNA molecule, the Stopper oligonucleotide is a DNA molecule, the Target is a DNA molecule, and the template-dependent polymerase is a DNA polymerase. In some aspects, the Primer oligonucleotide is an RNA molecule, the Stopper oligonucleotide is a DNA molecule, the Target is a DNA molecule, and the template-dependent polymerase is a DNA polymerase. In some aspects, the Primer oligonucleotide is a DNA molecule, the Stopper oligonucleotide is an RNA molecule, the Target is an RNA molecule, and the template-dependent polymerase is a reverse transcriptase. In some aspects, the Primer oligonucleotide is a DNA molecule, the Stopper oligonucleotide is a DNA molecule, the Target is an RNA molecule, and the template-dependent polymerase is a reverse transcriptase. In some aspects, the Primer oligonucleotide is an RNA molecule, the Stopper oligonucleotide is an RNA molecule, the Target is an RNA molecule, and the template-dependent polymerase is a reverse transcriptase. In some aspects, the Primer oligonucleotide is an RNA molecule, the Stopper oligonucleotide is an DNA molecule, the Target is an DNA molecule, and the template-dependent polymerase is an RNA polymerase.

In some aspects, the DNA polymerase is selected from the group consisting of Taq DNA polymerase, Bst DNA Polymerase, or DNA Polymerase I, Hemo Klen Taq, Phusion, Q5, T7 DNA polymerase, and KAPA HiFi. In some aspects, the reverse transcriptase is selected from the group consisting of Moloney Murine Leukemia Virus reverse transcriptase and Avian Myeloblastosis Virus reverse transcriptase.

In some aspects, the Target is a biological DNA or RNA molecule. In some aspects, the Target is obtained from a sample of cells, a biofluid, or a tissue. In some aspects, the biofluid is selected from the group consisting of blood, urine, saliva, cerebrospinal fluid, interstitial fluid, and synovial fluid. In some aspects, the tissue is a biopsy tissue or a surgically resected tissue.

In some aspects, the Target is a complementary DNA molecule generated through the reverse transcription of an RNA sample. In some aspects, the RNA sample is a biological RNA sample. In some aspects, the biological RNA sample is obtained from a human, animal, plant, or environmental specimen.

In some aspects, the Target is an amplicon DNA molecule generated through a DNA polymerase acting on a single-stranded DNA template. In some aspects, the amplicon DNA molecule is generated through multiple displacement amplification of a single cell DNA molecule.

In some aspects, the Target is a physically, chemically, or enzymatically generated product of a biological DNA molecule. In some aspects, the Target is the product of a fragmentation process. In some aspects, the fragmentation process is ultrasonication or enzymatic fragmentation.

In some aspects, the Target is the product of a bisulfite conversion reaction, an APOBEC (“apolipoprotein B mRNA editing enzyme, catalytic polypeptide-like”) reaction, a TAPS (TET-assisted pyridine borane sequencing) reaction, or other chemical or enzymatic reaction in which cytosine nucleotides are selectively converted to uracils based on methylation status.

In some aspects, the composition comprises a plurality of Stoppers and/or a plurality of Primers. In some aspects, each of the plurality of Stoppers share the same Fourth Sequence, each of which may share the same Third Sequence or have different Third Sequences. In some aspects, different Fourth Sequences may be present among the plurality of Stoppers, each of which may share the same Third Sequence or have different Third Sequences. As such, multiple Third Sequences and/or Fourth Sequences may be present among the plurality of Stoppers. In some aspects, the plurality of Stoppers having different Fourth Sequences are used, so as to bind to many different Targets. In some aspects, each of the plurality of Primers may share the same 3′ subsequence. In some aspects, each of the plurality of Primers may share the same 5′ subsequence. In some aspects, each of the plurality of Primers may comprise a different 3′ subsequence. In some aspects, multiple 3′ subsequences are present among the plurality of Primers, so as to bind to many different Targets. In some aspects, a plurality of identical Primers is used with a plurality of Stoppers having different Third and Fourth Sequences. In some aspects, a plurality of Primers having different 3′ subsequences are used with a plurality of Stoppers having identical Third and Fourth Sequences. In some aspects, a plurality of Primers having different 3′ subsequences are used with a plurality of Stoppers having different Third and Fourth Sequences are used, where each Target has a Primer-Stopper pair to generate a chimeric Amplicon from that Target. In this way, chimeric Amplicons may be generated from multiple Targets in a single, multiplex reaction. In some aspects, chimeric Amplicons may be generated from at least 2, 3, 4, 5, 6, 7, 8, 9, 10, 11, 12, 13, 14, 15, 20, 25, 30, 35, 40, 45, 50, 60, 70, 80, 90, 100, or more Targets in a single, multiplex reaction.

In one embodiment, provided herein are methods for generating a chimeric Amplicon comprising, from 5′ to 3′, a Primer Sequence, a Match-Complement Sequence, and a First-Complement Sequence, the method comprising: (a) mixing a Sample comprising a Target molecule comprising, from 5′ to 3′, a Binding Region, a Match Region, and a Priming Region with: (i) a template-dependent polymerase, (ii) a Primer oligonucleotide, wherein a 3′ subsequence of the Primer comprising at least 15 nucleotides is complementary to a Priming Region of the Target, and (iii) a Stopper oligonucleotide, wherein the Stopper comprises from 5′ to 3′: a First Sequence with a length between 5nt and 200nt, a Second Sequence with a length between 3nt and 50nt, a Loop Sequence with a length between 3nt and 70nt, and a Third Sequence with a length between 3nt and 50nt, wherein the Third Sequence is complementary to the Second Sequence and the Match Region of the Target, and a Fourth Sequence with a length between 6nt and 500nt, wherein the Fourth Sequence is complementary to the Binding Region of the Target, and (b) incubating the mixture at a temperature conducive to polymerase activity, wherein the Primer Sequence is homologous to the sequence of the Primer oligonucleotide, the Match-Complement Sequence is complementary to the Match Region of the Target, and the First-Complement Sequence is complementary to the First Sequence of the Stopper oligonucleotide. The complementarity relationships described above may be less than 100% complementarity. In some aspects, the complementarity relationships described above are ≥95%, ≥90%, ≥85%, or ≥80% complementarity. In some aspects, step (a) further comprises mixing the Sample with reagents and buffers needed for polymerase function. In some aspects, step (a) further comprises mixing the Sample with a fluorophore-functionalized DNA probe, optionally wherein the probe is a Taqman probe or a molecular beacon. In some aspects, step (a) further comprises mixing the Sample with a DNA intercalating dye, optionally wherein the dye comprises SybrGreen, EvaGreen, or Syto dyes.

In one embodiment, provided herein are methods for generating a chimeric Amplicon comprising, from 5′ to 3′, a Primer Sequence, a Match-Complement Sequence, and a First-Complement Sequence, the method comprising: (a) mixing a Sample comprising a Target molecule comprising, from 5′ to 3′, a Binding Region, a Match Region, and a Priming Region with: (i) a Primer oligonucleotide, wherein a 3′ subsequence of the Primer comprising at least 15 nucleotides is complementary to a Priming Region of the Target, and (ii) a Stopper oligonucleotide, wherein the Stopper comprises from 5′ to 3′: a First Sequence with a length between 5nt and 200nt, a Second Sequence with a length between 3nt and 50nt, a Loop Sequence with a length between 3nt and 70nt, and a Third Sequence with a length between 3nt and 50nt, wherein the Third Sequence is complementary to the Second Sequence and the Match Region of the Target, and a Fourth Sequence with a length between 6nt and 500nt, wherein the Fourth Sequence is complementary to the Binding Region of the Target, and (iii) an annealing buffer; (b) thermal annealing the mixture; (c) adding a template-dependent polymerase, reagents, and buffers needed for enzymatic function; and (d) incubating the mixture at a temperature conducive to polymerase activity, wherein the Primer Sequence is homologous to the sequence of the Primer oligonucleotide, the Match-Complement Sequence is complementary to the Match Region of the Target, and the First-Complement Sequence is complementary to the First Sequence of the Stopper oligonucleotide. The complementarity relationships described above may be less than 100% complementarity. In some aspects, the complementarity relationships described above are ≥95%, ≥90%, ≥85%, or ≥80% complementarity. In some aspects, step (b) comprises a thermocycling program of cooling from a temperature not lower than 78° C. to a temperature not higher than 25° C. In some aspects, the thermocycling program comprises steps that cool from about 78° C. to about 25° C., wherein the solution is held at each 5° C. temperature window for at least 5 minutes. In other words, for each 5 minutes of the thermocycling program, the program cools no faster than 5° C. per 5 minutes, i.e., spending≥5 minutes between 73° C. and 78° C., spending≥5 minutes between 68° C. and 73° C., etc. In some aspects, step (b) comprises incubating the mixture for between 10 minutes to 24 hours. In some aspects, step (b) comprises incubating the mixture at room temperature for between 10 minutes to 24 hours. In some aspects, step (a) or step (c) further comprises mixing the Sample with a fluorophore-functionalized DNA probe, optionally wherein the probe is a Taqman probe or a molecular beacon. In some aspects, step (a) or step (c) further comprises mixing the Sample with a DNA intercalating dye, optionally wherein the dye comprises SybrGreen, EvaGreen, or Syto dyes.

In some aspects, step (a) comprises mixing the sample with a composition according to any one of the present embodiments.

In some aspects, the Amplicon further comprises an Insert Sequence between the Primer Sequence and the Match-Complement Sequence.

In some aspects, the incubation occurs at a temperature between about 10° C. and about 74° C., between about 15° C. and about 74° C., between about 20° C. and about 74° C., between about 25° C. and about 74° C., between about 30° C. and about 74° C., between about 35° C. and about 74° C., between about 40° C. and about 74° C., between about 45° C. and about 74° C., between about 50° C. and about 74° C., between about 55° C. and about 74° C., between about 60° C. and about 74° C., between about 25° C. and about 65° C., between about 30° C. and about 65° C., between about 35° C. and about 65° C., or any range derivable therein. In some aspects, the incubation occurs at a temperature of about 10° C., 15° C., 20° C., 25° C., 30° C., 35° C., 40° C., 45° C., 50° C., 55° C., 60° C., 65° C., 70° C., or 74° C., or any value derivable therein. In some aspects, the incubation occurs for between 1 second and 20 hours, between 30 seconds and 20 hours, between 1 minute and 20 hours, between 2 minutes and 20 hours, between 5 minutes and 20 hours, between 10 minutes and 20 hours, between 30 minutes and 20 hours, between 60 minutes and 20 hours, between 2 hours and 20 hours, between 30 seconds and 2 hours, between 60 seconds and 2 hours, between 2 minutes and 2 hours, between 5 minutes and 2 hours, between 10 minutes and 2 hours, between 30 minutes and 2 hours, or any range derivable therein. In some aspects, the incubation occurs for at least 1 second, 10 seconds, 20 seconds, 30 seconds, 45 seconds, 60 seconds, 2 minutes, 5 minutes, 10 minutes, 20 minutes, 30 minutes, 40 minutes, 50 minutes, or 60 minutes and at most 20 hours, 15 hours, 10 hours, 5 hours, 2 hours, 1 hour, 50 minutes, 40 minutes, 30 minutes, 20 minutes, or 10 minutes. In some aspects, the incubation occurs for 1 second, 5 seconds, 10 seconds, 20 seconds, 30 seconds, 40 seconds, 50 seconds, 60 seconds, 2 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 14 hours, 16 hours, 18 hours, 20 hours, or any valuable derivable therein.

In some aspects, the incubation comprises thermal cycling alternating between a temperature higher than 78° C. (e.g., 78° C., 79° C., 80° C., 81° C., 82° C., 83° C., 84° C., 85° C., 86° C., 87° C., 88° C., 89° C., 90° C., 91° C., 92° C., 93° C., 94° C., or 95° C.) for between 1 second and 30 minutes (e.g., 1 second, 5 seconds, 10 seconds, 20 seconds, 30 seconds, 40 seconds, 50 seconds, 60 seconds, 2 minutes, 5 minutes, 10 minutes, 20 minutes, 30 minutes, or any value derivable therein) and a temperature not higher than 75° C. (e.g., 75° C., 74° C., 73° C., 72° C., 71° C., 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., 47° C., 46° C., or 45° C.) for between 1 second and 20 hours (e.g., 1 second, 5 seconds, 10 seconds, 20 seconds, 30 seconds, 40 seconds, 50 seconds, 60 seconds, 2 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 14 hours, 16 hours, 18 hours, 20 hours, or any valuable derivable therein). In some aspects, the methods further comprise at least 6, at least 7, at least 8, at least 9, at least 10, at least 11, at least 12, at least 13, at least 14, at least 15, at least 16, at least 17, at least 18, at least 19, or at least 20 additional thermal cycles.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Key reagent components of the chimeric Amplicon formation system. The dotted frame denotes the Stopper. The gray arrow on the right side of Primer and Stopper denotes the 3′ end of the oligonucleotide. The Stopper has, from 5′ to 3′, a First Sequence, a Second Sequence, a Loop Sequence, a Third Sequence, and a Fourth Sequence. The Second Sequence is complementary to the Third Sequence, the Loop Sequence is illustrated as an arc on the right of the hairpin. The system also includes a template-dependent polymerase.

FIG. 2: The chimeric Amplicon formation system includes a Target nucleic acid. Besides the components mentioned in the FIG. 1, the system also includes a Target nucleic acid. The Target has, from 5′ to 3′, a Binding Region, a Match Region, and a Priming Region. The gray arrow on the left side of Target denotes the 3′ end of the oligonucleotide. The Primer oligonucleotide and the Priming Region of the Target are complementary. The Fourth Sequence on the Stopper and the Binding Region on the Target are complementary. The Match Region is complementary to the Third Sequence of the Stopper.

FIG. 3: One embodiment of the Stopper. The Stopper can further comprise a Fifth Sequence between the Second Sequence and the Loop Sequence, and a Sixth Sequence between the Third Sequence and the Loop Sequence, where the Fifth Sequence is complementary to the Sixth Sequence.

FIG. 4: Mechanism of the induced template switching. From the 5′ to 3′ direction, the Target has a Priming Region (labeled P*), a Match Region (labeled M), and a Binding Region (labeled 4*), and the Stopper contains a First Sequence (labeled 1), a Second Sequence (labeled 2), a Loop Sequence (labeled L), a Third Sequence (labeled 3), and a Fourth Sequence (labeled 4). The Second Sequence is complementary to the Third Sequence, the Binding Sequence is complementary to Fourth Sequence, and the Match Region is homologous to the Second Sequence and complementary to the Third Sequence. There is a Target-Similar, with most regions identical to the Target, but the Match Region being replaced by region 5 on the Target-Similar. Region 5 is neither complementary to the Third Sequence nor homologous to the Second Sequence. On Stage 1, the Primer (P) is bound to the Priming Region of the Target, and the Fourth Sequence of the Stopper is bound to the Binding Region of the Target. The base pairs formed between the Target and the Stopper will form base stacks with the base pairs formed by the Stopper's hairpin, the stem of which is formed by the Second Sequence and the Third Sequence. As the polymerase extends the Primer to the Target-Stopper binding junction at Stage 2, the crossover geometry prevents the polymerase from further extending. On the left panel, because of the complementarity relationship between the Match Region and the Third Sequence, the multi-stranded molecule in Stage 2 can spontaneously rearrange via branch migration to the state shown in Stage 3, in which the 3′ end of the polymerase extension product bridges over the crossover junction and binds to the Second Sequence of the Stopper molecule. The polymerase is then able to continue extending in Stage 4, now recognizing the Stopper as the template. In Stage 5, the chimeric Amplicon finishes extension, and has a 5′ sequence complementary to the Target and a 3′ sequence complementary to the Stopper. On the right side of FIG. 4, since region 5 of Target-Similar is not complementary to the Third Sequence, the branch migration and rearrangement are unable to happen, thus the polymerase extension will stall at Stage 2 at the locus where the Stopper binds the Target-Similar.

FIGS. 5A-D: Experimental demonstration of induced template switching. (FIG. 5A) In reaction 1 (left panel), Target (SEQ ID NO: 1) was pre-annealed with the Primer (SEQ ID NO: 4) and Stopper (SEQ ID NO: 3). The Target has a Match Region that is complementary to the Third Sequence on the Stopper. In reaction 2 (right panel), the same Primer and Stopper were mixed with the Target-Similar (SEQ ID NO: 2) and the mixture was subjected to the annealing program. The Target-Similar does not have a Match Region that is complementary to the Third Sequence on the Stopper. Then, the same amount of T4 DNA polymerase and buffers were added into both reaction 1 and reaction 2, and the reactions incubated at 37° C. for 35 minutes, followed by 75° C. for 10 minutes. (FIGS. 5B&C) qPCR was used to quantitate both the amount of input DNA (the Target or Target-Similar), and the amount of PCR products formed through one polymerase extension reaction. The Primer and the Target-specific reverse primer (RP) (SEQ ID NO: 6) were used to determine the input qPCR cycle threshold (Cₜ). The product qPCR Cₜ value was determined based on the Primer and the Stopper-specific RP (SEQ ID NO: 5). (FIG. 5D) In the properly designed Target and Stopper system, the Cₜ value of the chimeric Amplicon was 14.7, similar to the Target Cₜ value of 14.4, indicating high efficiency of template switching. In the Target-Similar group where there was no Match Region that was complementary to the Third Sequence of the Stopper, the Cₜ value of the PCR product was 30.3, compared to the Target-Similar's 14.6. This 15.7 Cₜ value difference indicates that the efficiency of template switching is roughly 2\hat{ }⁽⁻15.7)=0.002% when the Target does not have a Match Region.

FIG. 6: Embodiment of chimeric Amplicon. Generated from the Chimeric Amplicon Formation system, the Amplicon has a Primer Sequence, a Match-Complement Sequence, and a First-Complement Sequence from the 5′ to 3′ direction. The Match-Complement Sequence is complementary to the Match Region on the Target, the First-Complement Sequence is complementary to the First Sequence on the Stopper.

FIG. 7: One embodiment for the chimeric Amplicon. The Amplicon could also have an Insert Sequence between the Primer Sequence and the Match-Complement Sequence that is complementary to the region between the Priming Region and the Match Region on the Target.

FIG. 8: Sanger validation of the chimeric Amplicon. The top embodiment shows the cartoon of design and the sequence details. After pre-annealing the DNA Primer (SEQ ID NO: 11), DNA Target (SEQ ID NO: 7), and the DNA Stopper (SEQ ID NO: 9) in 2×PBS buffer, the annealing product was incubated with T4 DNA polymerase at 37° C. for 35 minutes followed by 10 minutes at 75° C. Then, DNA Primer (SEQ ID NO: 11) and Sanger primer (SEQ ID NO: 12) were used to amplify the generated chimeric Amplicon (SEQ ID NO: 8). The PCR product was then Sanger sequenced, as the result shown at the bottom (SEQ ID NO: 10).

FIG. 9: Structures of the Stopper. As shown in the left cartoon, the x indicates the length of the stem sequence, the y is the length of the Loop Sequence, the z represents the number of loop nucleotides that are complementary to the sequence immediately to the 5′ of the Match Region of the Target. The table on the right side lists all the structures that have been tested with the results confirmed by Sanger Sequencing.

FIGS. 10A-C: Diversity of the Target sequence. Here, three different Target sequences (SEQ ID Nos: 32-34 in FIGS. 10A-C, respectively) that have different Priming Regions but the same Match Region sequence were tested. The three different Targets were annealed respectively with corresponding Primers (SEQ ID Nos: 35-37) and the Stopper (SEQ ID NO: 38), then incubated with either T4 DNA polymerase or T7 DNA polymerase and buffers needed at 37° C. for 35 minutes followed by 10 minutes at 75° C. The Sanger Sequencing results (SEQ ID Nos: 29-31) confirm the sequences of generated chimeric Amplicons, demonstrating the system is general and applicable to any Target sequence with the same Match Region.

FIG. 11: Demonstration of chimeric Amplicon formation in reverse transcription (RT). The top embodiment shows the cartoon of design and the sequence details. The RNA Stopper (SEQ ID NO: 40), DNA Primer (SEQ ID NO: 41), and RNA Target (SEQ ID NO: 39) were pre-annealed with RNase inhibitor in 2×PBS buffer. The annealing product was then mixed with dNTP and incubated at 65° C. for 4 minutes. Then, the reverse transcriptase, RNase inhibitor, and RT buffer were added into the reaction, and the mixture was subjected to 50° C. for 35 minutes and 85° C. for 5 minutes. The chimeric product (SEQ ID NO: 44) from the reverse transcription was amplified in PCR using the Sanger primer (SEQ ID NO: 42) and the DNA Primer (SEQ ID NO: 41), and its sequence was confirmed by Sanger Sequencing as shown in the bottom (SEQ ID NO: 43). The reverse transcriptase used here was Maxima H Minus Reverse Transcriptase.

FIG. 12: Library preparation. Primer can have a sequence at its 5′ region that is not complementary to the Target subsequence located to the 3′ of the Priming Region of the Target, which can be a Forward adaptor sequence. Likewise, the Fourth Sequence of the Stopper can comprise a Reverse adaptor sequence. Thus, the Amplicon will have appended sequencing adaptor sequences (or index sequences).

### DETAILED DESCRIPTION

Provided herein are materials and methods for formation of amplicons with chimeric sequence inherited from both a Target nucleic acid molecule and a Stopper nucleic acid molecule. These methods induce switching between the Target and the Stopper as the template during polymerase extension, achieving inheritance of information from both templates to the Amplicon simultaneously within only one cycle. The Stopper's sequence is rationally designed to comprise a hairpin with stem complementary to a region of the Target, and this relationship of sequence complementarity induces high-yield template switching. These methods are compatible with both thermostable and non-thermostable polymerases, thus the reaction is not limited in thermo-cycling but also feasible in an isothermal reaction. As such, using these methods, adapter sequences can be appended to both the 5′ and the 3′ ends of an amplicon molecule using a single isothermal polymerase extension step. The efficiency of the template switching has been experimentally demonstrated to be up to 100%. This method overcomes the limitation of thermostable polymerases and thermo-cycling reaction as well as low efficiency of ligation.

### I. CHIMERIC AMPLICON FORMATION

During polymerase extension of a DNA or RNA primer, the polymerase will add nucleotides to the 3′ end of the primer based on the sequence of the nucleic acid recognized as the template by the polymerase. Typically, the nucleic acid molecule recognized as the template by the polymerase does not change through the course of the polymerase extension process, even for very long amplicons over 5000nt.

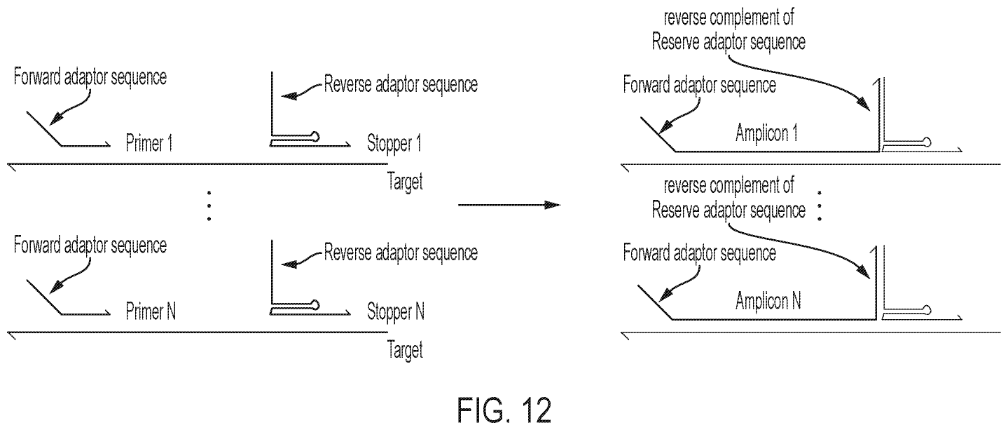

Provided herein are compositions and methods for inducing template switching by polymerases, which can result in polymerase extension products of a Primer (Amplicons) with a sequence that is a chimera between two distinct nucleic acid molecules. The Target, which serves as the initial template for the polymerase extension, is the nucleic acid molecule with which the Primer hybridizes. The Stopper oligonucleotide is rationally designed to hybridize to the Target sequence and have a pattern of sequence complementarities such that the extending polymerase switches to recognizing the Stopper as the template at the loci where the Target and the Stopper are bound. Any additional 5′ sequences on the Stopper are incorporated into the Amplicon, in addition to the sequences on the Target between, and including, the Priming Region and the Match Region. In some aspects, the additional 5′ sequences on the Stopper may comprise a sequencing adaptor or index sequence (FIG. 12).

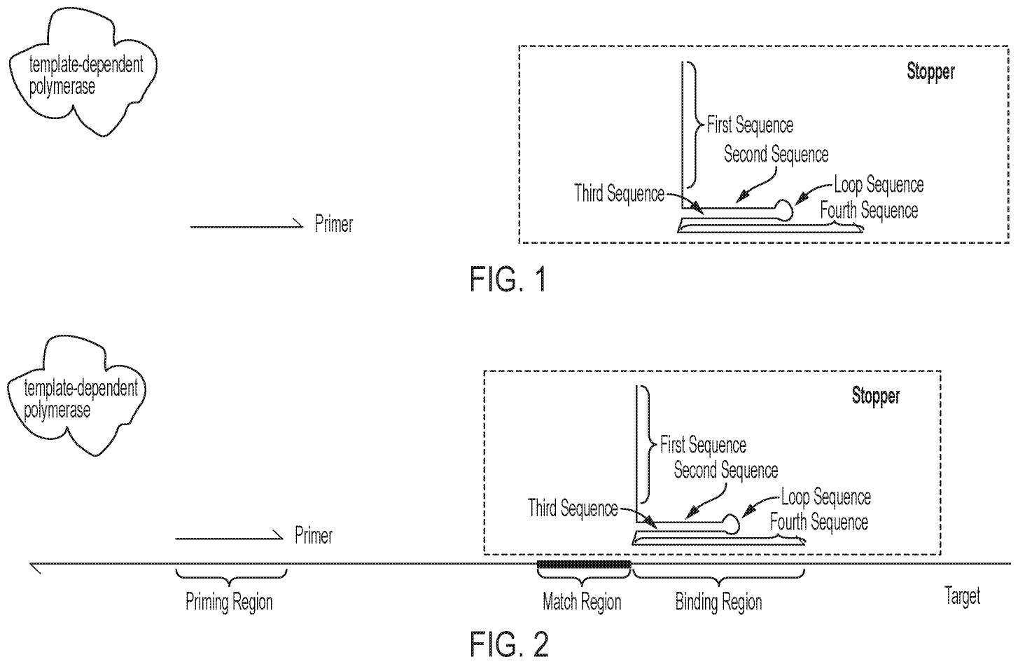

The Stopper is a nucleic acid species that comprises from 5′ to 3′ the following regions: A First Sequence, a Second Sequence, a Loop Sequence, a Third Sequence, and a Fourth Sequence. The Second Sequence and the Third Sequence form a hairpin stem; there is a Loop Sequence between the Second Sequence and the Third Sequence (FIG. 1).

The Target is a nucleic acid species that comprises from 5′ to 3′ the following regions: A Binding Region, a Match Region, and a Priming Region. The Fourth Sequence of the Stopper is complementary to the Binding Region. The Second Sequence of the Stopper is rationally designed to have the same sequence as the Match Region. The Match Region is complementary to the Third Sequence of the Stopper (FIG. 2). The Priming Region is complementary to the 3′ region of the Primer.

In some embodiments, the Primer has a sequence at its 5′ region that is not complementary to the Target subsequence located to the 3′ of the Priming Region of the Target. This sequence may comprise a sequencing adaptor or index sequence (FIG. 12).

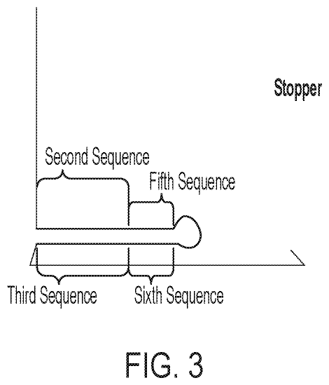

In some embodiments, the Stopper has a Fifth Sequence between the Second Sequence and the Loop Sequence, and a Sixth Sequence between the Third Sequence and the Loop Sequence, and the Fifth Sequence is complementary to the Sixth Sequence (FIG. 3). In some aspects, the Fifth Sequence is not complementary to a region of the Target nucleic acid positioned 3′ of the Match Region.

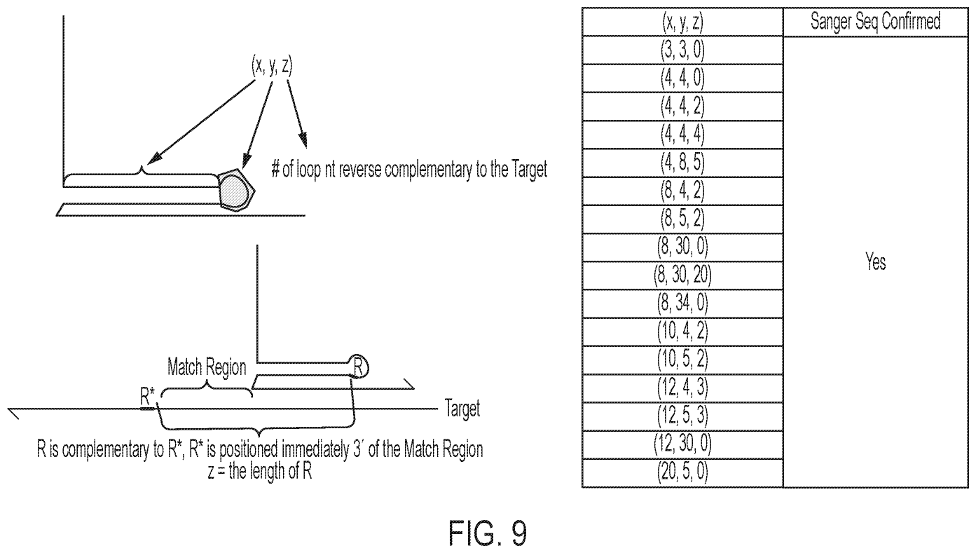

In some embodiment, a portion of the Loop Sequence is complementary to a region of the Target nucleic acid positioned immediately 3′ of the Match Region (FIG. 9). This portion of the Loop Sequence may have a length of 1nt, 2nt, 3nt, 4nt, 5nt, 6nt, 7nt, 8nt, 9nt, 10nt, 11nt, 12nt, 13nt, 14nt, 15nt, 16nt, 17nt, 18nt, 19nt, 20nt, or more.

Primers and Stoppers may be rationally designed in order to amplify desired target sequences, such as, for example, desired genes. To this end, the Primer sequence may be designed to be complementary to a sequence in the Target that is 3′ of the desired region (i.e., the Priming Region), and the Fourth Sequence of Stopper may be designed to be complementary to a sequence in the Target that is 5′ of the desired region (i.e., the Binding Region). Finally, the Third Sequence of the Stopper may be designed to be complementary to a sequence in the Target that is immediately 3′ of the Binding Region (i.e., the Match Region).

In some embodiments, the length of the Priming Region is between 15nt and 35nt, between 15nt and 30nt, between 15nt and 25nt, between 15nt and 20nt, between 20nt and 35nt, between 20nt and 30nt, between 20nt and 25nt, between 25nt and 35nt, between 25nt and 30nt, between 30nt and 35nt, or any range derivable therein. In some embodiments, the length of the Priming Region is 15nt, 16nt, 17nt, 18nt, 19nt, 20nt, 21nt, 22nt, 23nt, 24nt, 25nt, 26nt, 27nt, 28nt, 29nt, 30nt, 31nt, 32nt, 33nt, 34nt, or 35nt.

In some embodiments, the length of the Binding Region is between 6nt and 500nt, between 6nt and 400nt, between 6nt and 300nt, between 6nt and 200nt, between 6nt and 100nt, between 6nt and 75nt, between 6nt and 50nt, between 6nt and 25nt, between 6nt and 15nt, between 15nt and 500nt, between 15nt and 400nt, between 15nt and 300nt, between 15nt and 200nt, between 15nt and 100nt, between 15nt and 75nt, between 15nt and 50nt, between 15nt and 25nt, between 30nt and 500nt, between 30nt and 400nt, between 30nt and 300nt, between 30nt and 200nt, between 30nt and 100nt, between 30nt and 75nt, between 30nt and 50nt, or any range derivable therein. In some embodiments, the length of the Binding Region is at least 6nt, 7nt, 8nt, 9nt, 10nt, 11nt, 12nt, 13nt, 14nt, 15nt, 20nt, 25nt, 30nt, 40nt, 50nt, 60nt, 70nt, 80nt, 90nt, 100nt, 150nt, 200nt, 250nt, 300nt, 350nt, 400nt, or 450nt and at most 500nt, 450nt, 400nt, 350nt, 300nt, 250nt, 200nt, 150nt, 100nt, 90nt, 80nt, 70nt, 60nt, 50nt, 40nt, 30nt, 25nt, 20nt, or 15nt. In some embodiments, the length of the Binding Region is 6nt, 7nt, 8nt, 9nt, 10nt, 11nt, 12nt, 13nt, 14nt, 15nt, 20nt, 25nt, 30nt, 40nt, 50nt, 60nt, 70nt, 80nt, 90nt, 100nt, 150nt, 200nt, 250nt, 300nt, 350nt, 400nt, 450nt, or 500nt, or any value derivable therein.

In some embodiments, the length of the Match Region is between 3nt and 50nt, between 3nt and 40nt, between 3nt and 30nt, between 3nt and 25nt, between 3nt and 20nt, between 3nt and 15nt, between 3nt and 10nt, between 3nt and 5nt, between 5nt and 50nt, between 5nt and 40nt, between 5nt and 30nt, between 5nt and 25nt, between 5nt and 20nt, between 5nt and 15nt, between 5nt and 10nt, between 10nt and 50nt, between 10nt and 40nt, between 10nt and 30nt, between 10nt and 25nt, between 10nt and 20nt, between 10nt and 15nt, between 15nt and 50nt, between 15nt and 40nt, between 15nt and 30nt, between 15nt and 25nt, between 15nt and 20nt, between 20nt and 50nt, between 20nt and 40nt, between 20nt and 30nt, between 20nt and 25nt, or any range derivable therein. In some embodiments, the length of the Match Region is 3nt, 4nt, 5nt, 6nt, 7nt, 8nt, 9nt, 10nt, 11nt, 12nt, 13nt, 14nt, 15nt, 16nt, 17nt, 18nt, 19nt, 20nt, 21nt, 22nt, 23nt, 24nt, 25nt, 26nt, 27nt, 28nt, 29nt, 30nt, 31nt, 32nt, 33nt, 34nt, 35nt, 36nt, 37nt, 38nt, 39nt, 40nt, 41nt, 42nt, 43nt, 44nt, 45nt, 46nt, 47nt, 48nt, 49nt, or 50nt.

In some embodiments, the length of the First Sequence is between 5nt and 200nt, between 5nt and 150nt, between 5nt and 100nt, between 5nt and 75nt, between 5nt and 50nt, between 5nt and 25nt, between 5nt and 15nt, between 15nt and 200nt, between 15nt and 150nt, between 15nt and 100nt, between 15nt and 75nt, between 15nt and 50nt, between 15nt and 25nt, between 30nt and 200nt, between 30nt and 150nt, between 30nt and 100nt, between 30nt and 75nt, between 30nt and 50nt, or any range derivable therein. In some embodiments, the length of the First Sequence is at least 5nt, 6nt, 7nt, 8nt, 9nt, 10nt, 11nt, 12nt, 13nt, 14nt, 15nt, 20nt, 25nt, 30nt, 40nt, 50nt, 60nt, 70nt, 80nt, 90nt, 100nt, 150nt, or 175nt and at most 200nt, 150nt, 100nt, 90nt, 80nt, 70nt, 60nt, 50nt, 40nt, 30nt, 25nt, 20nt, or 15nt. In some embodiments, the length of the First Sequence is 5nt, 6nt, 7nt, 8nt, 9nt, 10nt, 11nt, 12nt, 13nt, 14nt, 15nt, 20nt, 25nt, 30nt, 40nt, 50nt, 60nt, 70nt, 80nt, 90nt, 100nt, 110nt, 120nt, 130nt, 140nt, 150nt, 160nt, 170nt, 180nt, 190nt, 200nt, or any value derivable therein.

In some embodiments, the length of the Second Sequence is between 3nt and 50nt, between 3nt and 40nt, between 3nt and 30nt, between 3nt and 25nt, between 3nt and 20nt, between 3nt and 15nt, between 3nt and 10nt, between 3nt and 5nt, between 5nt and 50nt, between 5nt and 40nt, between 5nt and 30nt, between 5nt and 25nt, between 5nt and 20nt, between 5nt and 15nt, between 5nt and 10nt, between 10nt and 50nt, between 10nt and 40nt, between 10nt and 30nt, between 10nt and 25nt, between 10nt and 20nt, between 10nt and 15nt, between 15nt and 50nt, between 15nt and 40nt, between 15nt and 30nt, between 15nt and 25nt, between 15nt and 20nt, between 20nt and 50nt, between 20nt and 40nt, between 20nt and 30nt, between 20nt and 25nt, or any range derivable therein. In some embodiments, the length of the Second Sequence is 3nt, 4nt, 5nt, 6nt, 7nt, 8nt, 9nt, 10nt, 11nt, 12nt, 13nt, 14nt, 15nt, 16nt, 17nt, 18nt, 19nt, 20nt, 21nt, 22nt, 23nt, 24nt, 25nt, 26nt, 27nt, 28nt, 29nt, 30nt, 31nt, 32nt, 33nt, 34nt, 35nt, 36nt, 37nt, 38nt, 39nt, 40nt, 41nt, 42nt, 43nt, 44nt, 45nt, 46nt, 47nt, 48nt, 49nt, or 50nt.

In some embodiments, the length of the Loop Sequence is between 3nt and 70nt, between 3nt and 60nt, between 3nt and 50nt, between 3nt and 40nt, between 3nt and 30nt, between 3nt and 25nt, between 3nt and 20nt, between 3nt and 15nt, between 3nt and 10nt, between 3nt and 5nt, between 5nt and 70nt, between 5nt and 60nt, between 5nt and 50nt, between 5nt and 40nt, between 5nt and 30nt, between 5nt and 25nt, between 5nt and 20nt, between 5nt and 15nt, between 5nt and 10nt, between 10nt and 70nt, between 10nt and 60nt, between 10nt and 50nt, between 10nt and 40nt, between 10nt and 30nt, between 10nt and 25nt, between 10nt and 20nt, between 10nt and 15nt, between 15nt and 70nt, between 15nt and 60nt, between 15nt and 50nt, between 15nt and 40nt, between 15nt and 30nt, between 15nt and 25nt, between 15nt and 20nt, between 20nt and 70nt, between 20nt and 60nt, between 20nt and 50nt, between 20nt and 40nt, between 20nt and 30nt, between 20nt and 25nt, or any range derivable therein. In some embodiments, the length of the Loop Sequence is 3nt, 4nt, 5nt, 6nt, 7nt, 8nt, 9nt, 10nt, 11nt, 12nt, 13nt, 14nt, 15nt, 16nt, 17nt, 18nt, 19nt, 20nt, 21nt, 22nt, 23nt, 24nt, 25nt, 26nt, 27nt, 28nt, 29nt, 30nt, 31nt, 32nt, 33nt, 34nt, 35nt, 36nt, 37nt, 38nt, 39nt, 40nt, 41nt, 42nt, 43nt, 44nt, 45nt, 46nt, 47nt, 48nt, 49nt, 50nt, 51nt, 52nt, 53nt, 54nt, 55nt, 56nt, 57nt, 58nt, 59nt, 60nt, 61nt, 62nt, 63nt, 64nt, 65nt, 66nt, 67nt, 68nt, 69nt, or 70nt.

In some embodiments, the length of the Third Sequence is between 3nt and 50nt, between 3nt and 40nt, between 3nt and 30nt, between 3nt and 25nt, between 3nt and 20nt, between 3nt and 15nt, between 3nt and 10nt, between 3nt and 5nt, between 5nt and 50nt, between 5nt and 40nt, between 5nt and 30nt, between 5nt and 25nt, between 5nt and 20nt, between 5nt and 15nt, between 5nt and 10nt, between 10nt and 50nt, between 10nt and 40nt, between 10nt and 30nt, between 10nt and 25nt, between 10nt and 20nt, between 10nt and 15nt, between 15nt and 50nt, between 15nt and 40nt, between 15nt and 30nt, between 15nt and 25nt, between 15nt and 20nt, between 20nt and 50nt, between 20nt and 40nt, between 20nt and 30nt, between 20nt and 25nt, or any range derivable therein. In some embodiments, the length of the Third Sequence is 3nt, 4nt, 5nt, 6nt, 7nt, 8nt, 9nt, 10nt, 11nt, 12nt, 13nt, 14nt, 15nt, 16nt, 17nt, 18nt, 19nt, 20nt, 21nt, 22nt, 23nt, 24nt, 25nt, 26nt, 27nt, 28nt, 29nt, 30nt, 31nt, 32nt, 33nt, 34nt, 35nt, 36nt, 37nt, 38nt, 39nt, 40nt, 41nt, 42nt, 43nt, 44nt, 45nt, 46nt, 47nt, 48nt, 49nt, or 50nt.

In some embodiments, the length of the Fourth Sequence is between 6nt and 500nt, between 6nt and 400nt, between 6nt and 300nt, between 6nt and 200nt, between 6nt and 100nt, between 6nt and 75nt, between 6nt and 50nt, between 6nt and 25nt, between 6nt and 15nt, between 15nt and 500nt, between 15nt and 400nt, between 15nt and 300nt, between 15nt and 200nt, between 15nt and 100nt, between 15nt and 75nt, between 15nt and 50nt, between 15nt and 25nt, between 30nt and 500nt, between 30nt and 400nt, between 30nt and 300nt, between 30nt and 200nt, between 30nt and 100nt, between 30nt and 75nt, between 30nt and 50nt, or any range derivable therein. In some embodiments, the length of the Fourth Sequence is at least 6nt, 7nt, 8nt, 9nt, 10nt, 11nt, 12nt, 13nt, 14nt, 15nt, 20nt, 25nt, 30nt, 40nt, 50nt, 60nt, 70nt, 80nt, 90nt, 100nt, 150nt, 200nt, 250nt, 300nt, 350nt, 400nt, or 450nt and at most 500nt, 450nt, 400nt, 350nt, 300nt, 250nt, 200nt, 150nt, 100nt, 90nt, 80nt, 70nt, 60nt, 50nt, 40nt, 30nt, 25nt, 20nt, or 15nt. In some embodiments, the length of the Fourth Sequence is 6nt, 7nt, 8nt, 9nt, 10nt, 11nt, 12nt, 13nt, 14nt, 15nt, 20nt, 25nt, 30nt, 40nt, 50nt, 60nt, 70nt, 80nt, 90nt, 100nt, 150nt, 200nt, 250nt, 300nt, 350nt, 400nt, 450nt, or 500nt, or any value derivable therein.

In some embodiments, the complementarity relationships described above are not necessarily 100% complementarity. In some embodiments, the complementarity relationships described above are ≥95%, ≥90%, ≥85%, or ≥80% complementarity. In other words, if a first sequence is defined as being complementary to a second sequence, then the reverse complement of the first sequence may be 100%, ≥95%, ≥90%, ≥85%, or ≥80% identical to the second sequence. By way of example, if one sequence is defined as being at least 80% complementary to another sequence, then that sequence is at least 80% identical to the reverse complement of the other sequence.

“Identity” or “homology” refers to sequence similarity between two nucleic acid molecules. Identity can be determined by comparing a corresponding position in each sequence or by comparing an alignment of the sequences being compared. When a position in the compared sequences is occupied by the same base, then the molecules are identical at that position. A degree of identity between sequences can be a function of the number of matching or homologous positions shared by the sequences. “Unrelated” or “non-complementary” sequences share less than 40% identity, or alternatively less than 25% identity. Sequence identity can refer to a % identity of one sequence to another sequence. As a practical matter, when sequences are defined as being complementary, then the reverse complement of one of the sequences will be at least 50%, at least 60%, at least 70%, at least 80%, at least 85%, at least 90%, at least 92%, at least 95%, at least 96%, at least 97%, at least 98%, at least 99%, or 100% identical to the other sequence. One particular example of algorithms that are suitable for determining percent sequence identity is the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, to determine percent sequence identity for two or more polynucleotide sequences. Software for performing BLAST analyses is publicly available through the National Center for Biotechnology Information.

In some embodiments, two nucleic acid molecules are complementary if they can hybridize with each other under stringent conditions. As used herein “stringent conditions” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. By way of example, stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the sequence(s), the charge composition of the nucleic acid(s), and to the presence or concentration of solvent(s) in a hybridization mixture. It is also understood that these ranges, compositions and conditions for hybridization are mentioned by way of non-limiting examples only, and that the desired stringency for a particular hybridization reaction is often determined empirically by comparison to one or more positive or negative controls. In some embodiments, two nucleic acid molecules are complementary if they can hybridize with each other under low stringency conditions. Non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suit a particular application. In some embodiments, two nucleic acid molecules are non-complementary if they are unable to hybridize with each other under low stringency conditions.

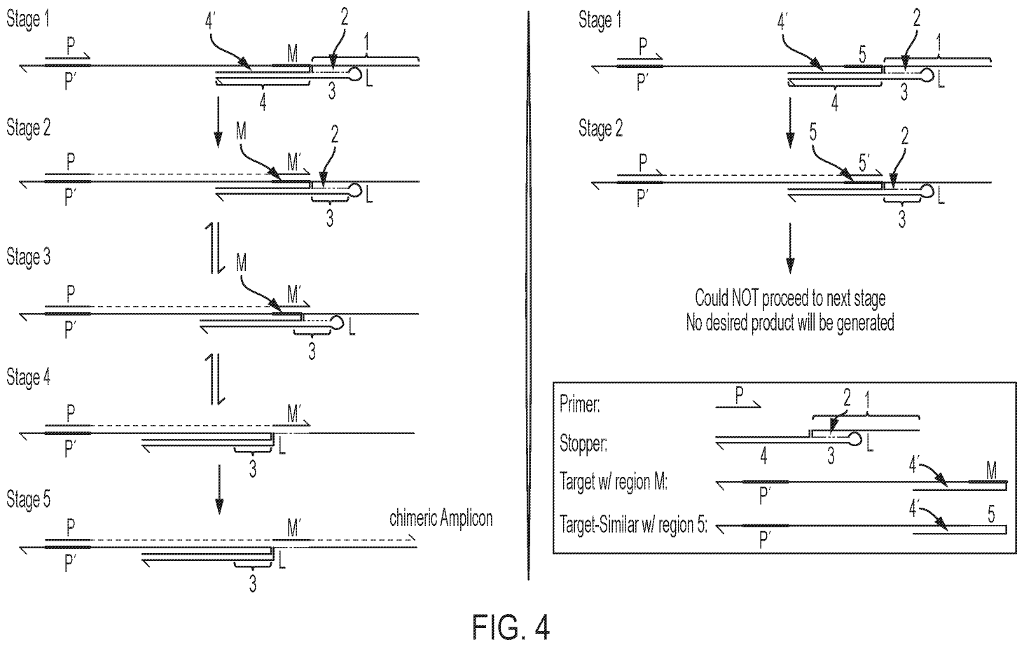

The mechanism of induced template switching is shown in FIG. 4. At Stage 1, the Primer is bound to the Target. The base pairs formed between the Target and the Stopper at the Match Region (region M) will form base stacks with the base pairs formed by the Stopper's hairpin comprising the Second Sequence (region 2) and the Third Sequence (region 3). When the polymerase recognizes the Target as the template and extends the Primer to the Target-Stopper binding junction in Stage 2, the polymerase finishes extending on the Match Region (region M) but is unable to further extend, due to the crossover geometry present.

Because of the complementarity relationship between the Match Region (region M) and the Third Sequence (region 3), the multi-stranded molecule in Stage 2 can spontaneously rearrange via branch migration to the state shown in Stage 3, in which the 3′ end of the polymerase extension product bridges over the crossover junction and binds to the Second Sequence (region 2) of the Stopper molecule. The polymerase is then able to continue extending in Stage 4, now recognizing the Stopper as the template. In Stage 5, the chimeric Amplicon finishes extension, and has its 5′ sequence dependent on the Target and its 3′ sequence dependent on the Stopper.

On the right side of FIG. 4, an alternative system is shown where the Third Sequence (region 3) of the Stopper is not complementary to the region 5 of the Target-Similar, thus the rearrangement from Stage 2 to Stage 3 is not possible, and polymerase extension stalls at Stage 2 at the locus where the Stopper binds the Target.

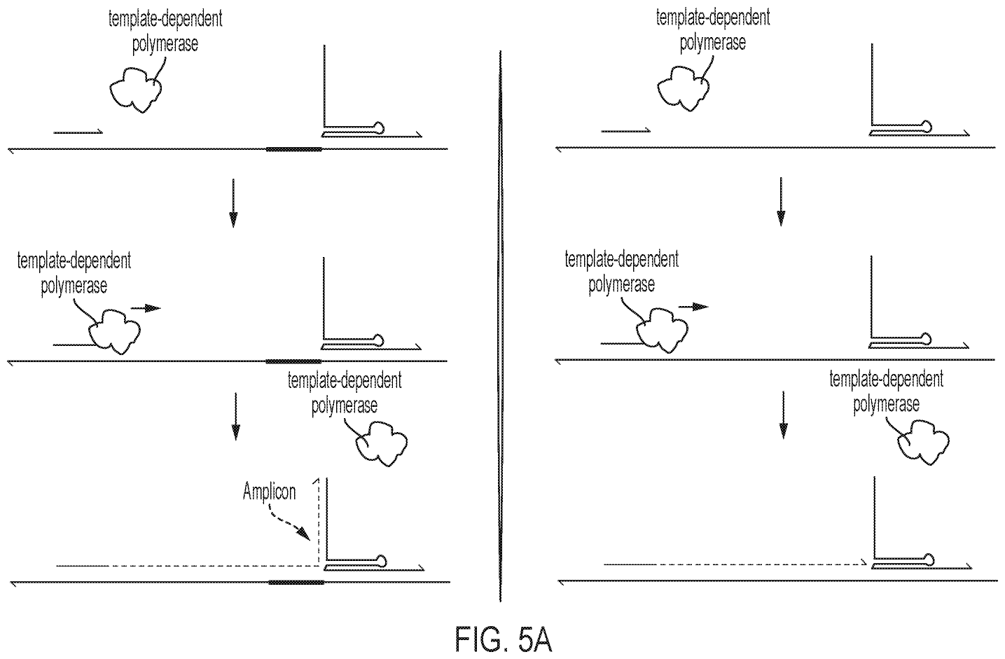

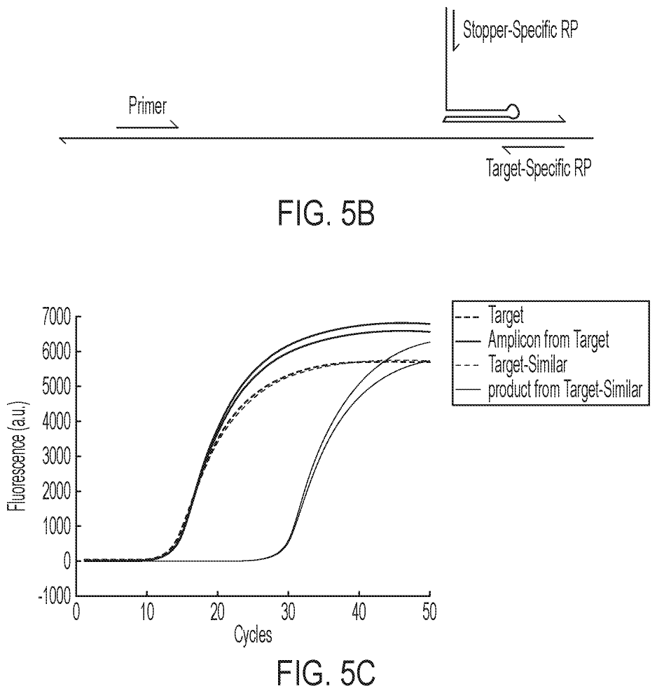

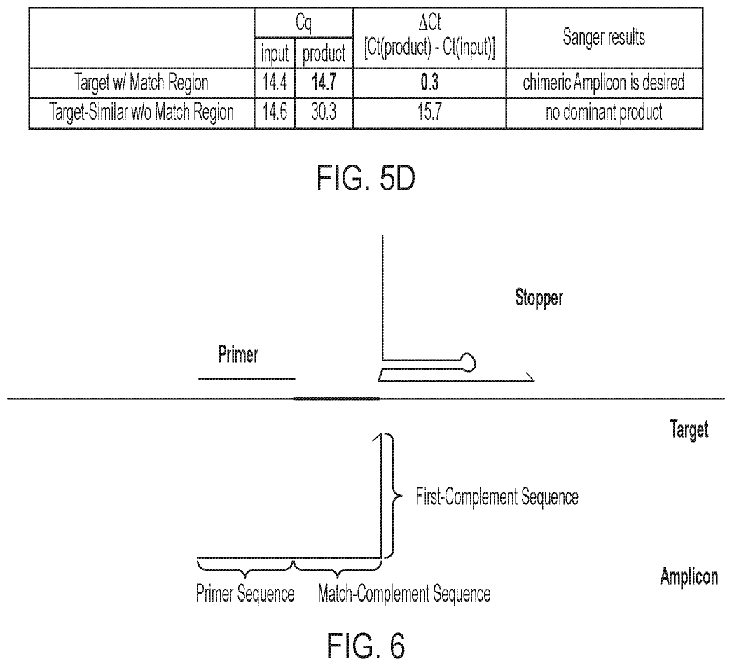

FIG. 5 shows the experimental demonstration of induced template switching. qPCR was used to quantitate both the amount of Target and Target-Similar oligonucleotides introduced to the reaction initially, and the amounts of products formed through one polymerase extension reaction. The only difference between the Target and Target-Similar is whether there is a Match Region that is complementary to the Third Sequence of the Stopper. The qPCR cycle threshold (Cₜ) value of the Target or Target-Similar was determined based on the DNA Primer and the Target-specific reverse primer. The product qPCR Cₜ value was determined based on the Primer and the Stopper-specific reverse primer. In the properly designed Target and Stopper system, the Cₜ value of the chimeric Amplicon was 14.7, similar to the Target Cₜ value of 14.4, indicating high efficiency of template switching. In the negative control system, where the Target-Similar does not have a region that is complementary to the Stopper's Third Sequence, the Cₜ value of the product was 30.3, compared to the Target-Similar's 14.6. This 15.7 Cₜ value difference indicates that the efficiency of template switching is roughly 2\hat{ }(−15.7)=0.002% if the Target does not comprise a Match Region complementary to the Third Sequence of Stopper.

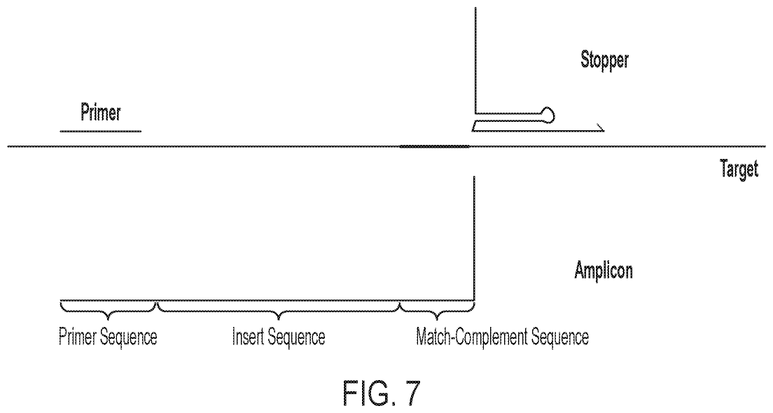

The Amplicon generated from the reaction has, from 5′ to 3′, a Primer Sequence, a Match-Complement Sequence, and a First-Complement Sequence (FIG. 6). In some embodiments, the Amplicon has an Insert Sequence between the Primer Sequence and the Match-Complement Sequence (FIG. 7). The Insert Sequence and the Match-Complement Sequence are complementary to regions of the Target, and the First-Complement sequence is complementary to regions of the Stopper.

Appending adaptors to the 5′ and 3′ ends of target nucleic sequences is an important step. For example, linking sequencing adaptors to the 5′ and 3′ ends of target nucleic sequences is necessary for high-throughput sequencing like Next Generation Sequencing (NGS) or Third Generation Sequencing. In some embodiments, the Primer has a 5′ AdapterA sequence. In some embodiments, the First Sequence has a 5′ AdapterB sequence. In some embodiments, the chimeric Amplicon has the AdapterA sequence at its 5′ end and the complement of the AdapterB sequence at its 3′ end. In some embodiments, the AdapterA and AdapterB sequences comprise sequencing adapters for high-throughput sequencing.

### II. POTENTIAL REDUCTION OF PRIMER DIMERS

In highly multiplexed PCR reactions, the number of unwanted primer dimer species formed through nonspecific binding of primers can be a significant barrier to scaling. In the provided template switching approach, the Stoppers are rationally designed and can comprise sequences or chemical modifications at the 3′ end that prevent polymerase extension. Consequently, the amount of primer dimers formed can be lower than in traditional multiplex PCR reactions.

For example, in a typical N-plex PCR reaction, 2N kinds of primer are used, and any two primers could form a primer dimer. The possibility of primer-dimer rate is 2N*(2N−1)/2. If the 3′ ends of these primers are modified to make them un-extensible, the primers are less likely to form primer dimers, but they will also lose function and cannot not extend on the Target. In the present methods, the Stoppers may be modified to make them un-extensible without influencing the generation of chimeric Amplicon. As such, there would be only N primers capable of forming primer-dimer, the chance will be reduced to N*(N−1)/2. In this way, the primer-dimer rate is reduced by approximately 4-fold.

### III. EXEMPLARY EXPERIMENTAL PROTOCOLS AND CONDITIONS

### A. Experimental Protocols and Conditions for Chimeric Amplicon Generation in Polymerase Chain Reaction (PCR)

For the experimental results presented herein, the final concentration of the Primer, Target, and the Stopper were each 3 nM in 50 μL of reaction mixture unless otherwise noted. Either T4 DNA polymerase (Thermo Fisher) or T7 DNA polymerase (Thermo Fisher) as well as buffers and reagents needed for the enzymatic function were used. Thermal cycling was performed using either a Bio-Rad T100 or a Bio-Rad C1000 instrument. The thermal cycling protocol was as follows:
Lid temperature: 90° C.
1.37° C. 35 minutes.
2.75° C. 10 minutes.

### B. Experimental Protocols and Conditions for Chimeric Amplicon Generation in Reverse Transcription

For the experimental results presented herein, the final concentration of the Primer, Target and the Stopper were each 0.5 nM in 50 μL of reaction mixture unless otherwise noted. Maxima H Minus Reverse Transcriptase (Thermo Fisher) was used for the reverse transcription reaction. Thermal cycling was performed using either a Bio-Rad T100 or a Bio-Rad C1000 instrument. The detailed protocol and the thermal cycling protocol were as follows:
1. Mix the dNTPs, Target, Stopper, and the Primers together, incubate at 65° C. for 4 minutes, then quickly chill on ice.
2. Add 500 units of Maxima H Minus Reverse Transcriptase, and 120 units of RiboLock RNase Inhibitor (Thermo Fisher) and the buffers needed for enzymatic function, then incubate at 50° C. for 35 minutes, followed by 85° C. for 5 minutes.

### C. Annealing Experimental Protocols and Conditions

For the experimental results presented herein, the final concentration of the Primer, Target and the Stopper were each 3 nM in 50 μL of reaction mixture unless otherwise noted. Unless otherwise noted, 2× Phosphate-buffered saline (PBS) buffer was used for all experiments. Thermal cycling was performed using an Eppendorf Mastercycler. The thermal cycling protocol was as follows:
1. 95° C. 2 minutes.
2. Then the program cools from 95° C. to 20° C. at the ramp speed of 0.01° C. per 6 seconds.

When any of the starting materials was RNA or contained RNA nucleotides, 160 units of RNase inhibitor were added into the mixture prior to the annealing.

### D. Experimental Protocols and Conditions for Preparation for Sanger Sequencing

For the Sanger Sequencing results presented herein, a pair of primers, i.e., the Primer and the Stopper-Specific RP, were used to amplify the chimeric Amplicon to achieve the minimum amount necessary for Sanger Sequencing. The final concentration of each primer was 400 nM in 10 μL of reaction mixture. Unless otherwise noted, the PowerUp SYBR DNA Polymerase Mastermix (Thermo Fisher) was used for all the experiments. Thermal cycling and fluorescence measurement were performed using a Bio-Rad CFX96 qPCR instrument. The thermal cycling protocol was as follows:
1. 95° C. 3 minutes.
2. 55 cycles of (95° C. for 10 seconds, 60° C. for 30 seconds)

Fluorescent signals were all collected under 60° C.

### IV. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase, although RNA polymerase and reverse transcriptase are also contemplated. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

“Incorporating,” as used herein, means becoming part of a nucleic acid polymer.

The term “in the absence of exogenous manipulation” as used herein refers to there being modification of a nucleic acid molecule without changing the solution in which the nucleic acid molecule is being modified. In specific embodiments, it occurs in the absence of the hand of man or in the absence of a machine that changes solution conditions, which may also be referred to as buffer conditions. However, changes in temperature may occur during the modification.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” “Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.” In addition, the term “adaptor” can indicate a linear adaptor (either single stranded or double stranded) or a stem-loop adaptor. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

A “nucleic acid molecule” or “nucleic acid target molecule” refers to any single-stranded or double-stranded nucleic acid molecule including standard canonical bases, hypermodified bases, non-natural bases, or any combination of the bases thereof. For example and without limitation, the nucleic acid molecule contains the four canonical DNA bases—adenine, cytosine, guanine, and thymine, and/or the four canonical RNA bases—adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin. Examples of nucleic acid molecules include genomic DNA, cDNA, RNA, a DNA/RNA hybrid, amplified DNA, a pre-existing nucleic acid library, etc. A nucleic acid may be obtained from a human sample, such as blood, serum, plasma, cerebrospinal fluid, cheek scrapings, biopsy, semen, urine, feces, saliva, sweat, etc. A nucleic acid molecule may be subjected to various treatments, such as repair treatments and fragmenting treatments. Fragmenting treatments include mechanical, sonic, and hydrodynamic shearing. Repair treatments include nick repair via extension and/or ligation, polishing to create blunt ends, removal of damaged bases, such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

The term “non-complementary” refers to nucleic acid sequence that lacks the ability to form at least one Watson-Crick base pair through specific hydrogen bonds.

The term “degenerate” as used herein refers to a nucleotide or series of nucleotides wherein the identity can be selected from a variety of choices of nucleotides, as opposed to a defined sequence. In specific embodiments, there can be a choice from two or more different nucleotides. In further specific embodiments, the selection of a nucleotide at one particular position comprises selection from only purines, only pyrimidines, or from non-pairing purines and pyrimidines.

The term “secondary structure” as used herein refers to the set of interactions between bases pairs. For example, in a DNA double helix, the two strands of DNA are held together by hydrogen bonds. The secondary structure is responsible for the shape that the nucleic acid assumes. For a single stranded nucleic acid, the simplest secondary structure is linear. For a linear secondary structure, no two subsequences of a nucleic acid molecule form an intramolecular structure stronger than −2 kcal/mol. As another example for a single stranded nucleic acid, one portion of the nucleic acid molecule may hybridize with a second portion of the same nucleic acid molecule, thereby forming a hairpin to stem loop secondary structure. For a non-linear secondary structure, at least two subsequences of a nucleic acid molecule from an intramolecular structure stronger than −2 kcal/mol.

A “Target” for a chimeric amplification system described herein can be any single-stranded nucleic acid, such as single-stranded DNA and single-stranded RNA, including double-stranded DNA and RNA rendered single-stranded through heat shock, asymmetric amplification, competitive binding, and other methods standard to the art. A DNA Target may be the product of RNA subjected to reverse transcription. In some instances, a Target may be a mixture (chimera) of DNA and RNA. In other instances, a Target comprises artificial nucleic acid analogs. In some instances, a Target may be naturally occurring (e.g., genomic DNA) or it may be synthetic (e.g., from a genomic library). As used herein, a “naturally occurring” nucleic acid sequence is a sequence that is present in nucleic acid molecules of organisms or viruses that exist in nature in the absence of human intervention or that is present in any biological sample. In some instances, a Target is genomic DNA, messenger RNA, ribosomal RNA, cell-free DNA, micro-RNA, pre-micro-RNA, pro-micro-RNA, long non-coding RNA, small RNA, epigenetically modified DNA, epigenetically modified RNA, viral DNA, viral RNA or piwi-RNA. In certain instances, a Target nucleic acid is a nucleic acid that naturally occurs in an organism or virus. In some instances, a Target nucleic is the nucleic acid of a pathogenic organism or virus. In certain instances, a Target of interest is linear, while in other instances, a Target is circular (e.g., plasmid DNA, mitochondrial DNA, or plastid DNA).

In certain instances, a Target nucleic acid molecule of interest is about 19 to about 1,000,000 nucleotides (nt) in length. In some instances, the Target is about 19 to about 100, about 100 to about 1000, about 1000 to about 10,000, about 10,000 to about 100,000, or about 100,000 to about 1,000,000 nucleotides in length. In some instances, the Target is about 20, about 100, about 200, about 300, about 400, about 500, about 600, about 700, about 800, about 900, about 1,000, about 2,000, about 3,000, about 4,000, about 5,000, about 6,000, about 7,000, about 8,000, about 9000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, or about 1,000,000 nucleotides in length. It is to be understood that the Target nucleic acid may be provided in the context of a longer nucleic acid (e.g., such as a coding sequence or gene within a chromosome or a chromosome fragment).

“Biological sample” means a material obtained or isolated from a fresh or preserved biological sample or synthetically created source that contains nucleic acids of interest. Samples can include at least one cell, fetal cell, cell culture, tissue specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals, other animals, plants, fungi, bacteria, and viruses.

As used herein in relation to a nucleotide sequence, “substantially known” refers to having sufficient sequence information in order to permit preparation of a nucleic acid molecule, including its amplification. This will typically be about 100%, although in some embodiments some portion of an adaptor sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

### V. KITS

The technology described herein includes kits comprising Stopper oligonucleotides as disclosed herein, and optionally Primers. Exemplary kits include qPCR kits, Sanger kits, NGS panels, and nanopore sequencing panels. A “kit” refers to a combination of physical elements. For example, a kit may include, for example, one or more components such as nucleic acid Primers, nucleic acid Stoppers, enzymes, reaction buffers, an instruction sheet, and other elements useful to practice the technology described herein. These physical elements can be arranged in any way suitable for carrying out the invention.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow molded plastic containers into which the desired vials are retained. A kit will also include instructions for employing the kit components as well the use of any other reagent not included in the kit. Instructions may include variations that can be implemented.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Chimeric Amplicon Formation in PCR

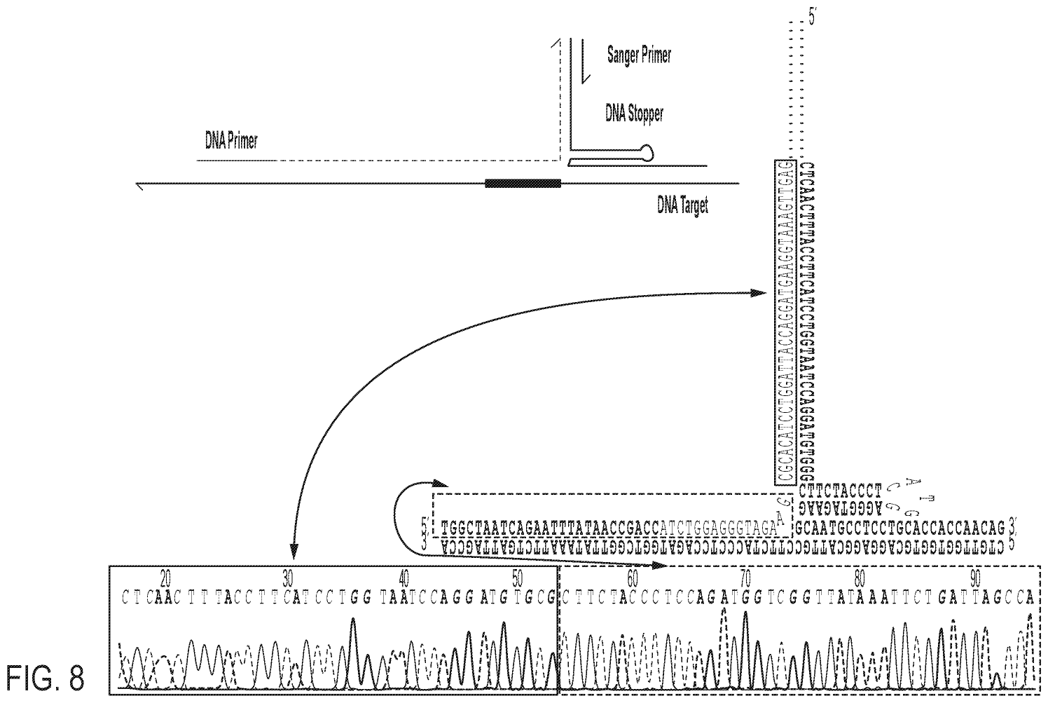

FIG. 8 shows an experimental demonstration using the DNA Primer, DNA Stopper, and the DNA Target oligonucleotides to generate the chimeric Amplicon. The three oligonucleotides were first annealed in 2×PBS buffer, then the annealed product was incubated with T4 DNA polymerase at 37° C. for 35 minutes followed by 10 minutes at 75° C. The Primer and the Sanger primer were used to amplify the generated chimeric Amplicon to achieve the minimum input amount requirement for Sanger Sequencing. Sequencing result was shown in the bottom of FIG. 8, confirming the sequence of the chimeric Amplicons.

FIG. 9 presents a cartoon in the left panel, where the x is the length of stem sequence of the hairpin, the y is the length of loop sequence of the hairpin, the z stands for the number of nucleotides on the loop that is reverse complementary to the Target. All the experimentally tested structures of Stoppers were listed in the table in FIG. 9 and their respective sequences are SEQ ID Nos: 13-28.

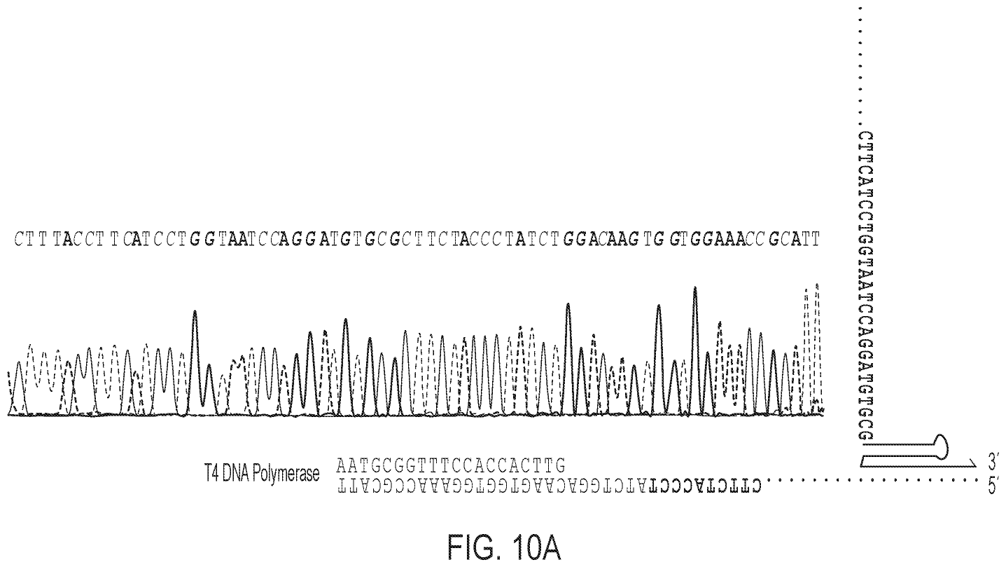

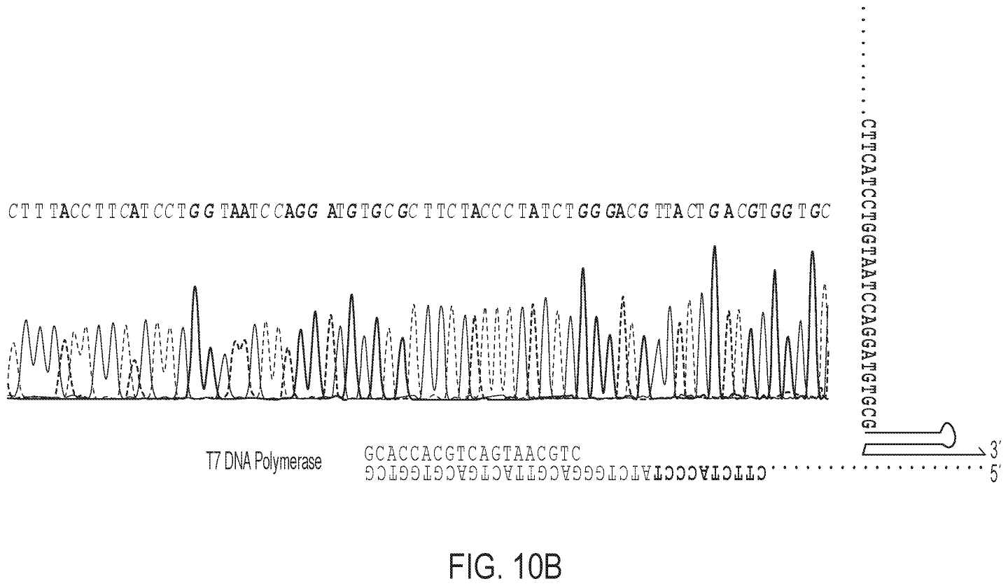

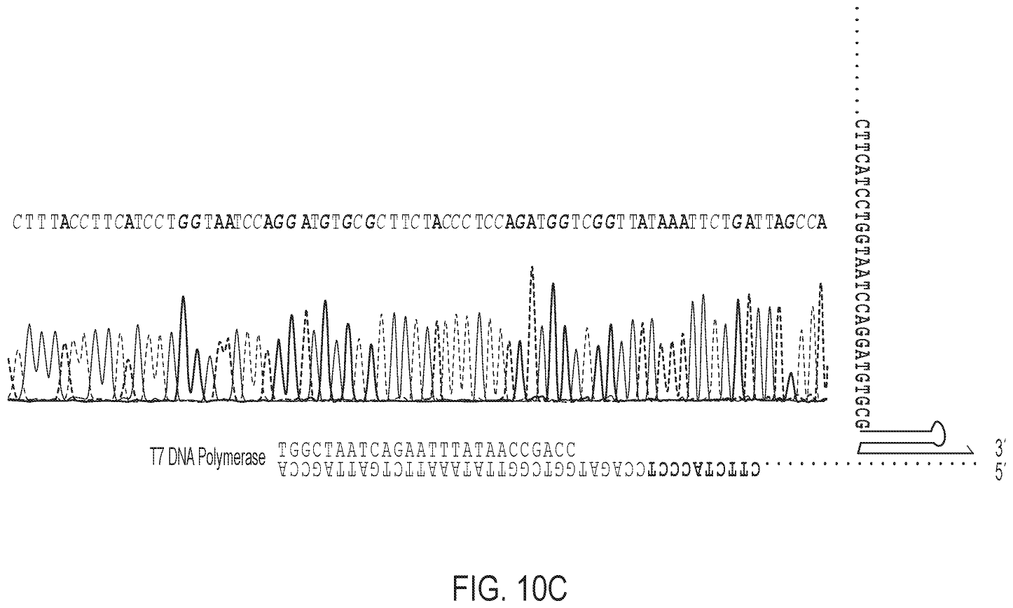

Compatibility of different Target with the same Match Region was validated in FIG. 10. Target with different Priming Sequences, and Insert Sequences were mixed with the same Stopper, the Sanger Sequencing products proves the feasibility.

### Example 2—Chimeric Amplicon Formation in Reverse Transcription

The Chimeric Amplicon Formation system is not limited only in reaction using DNA polymerase, but also could be used in reactions using reverse transcription or RNA polymerase.

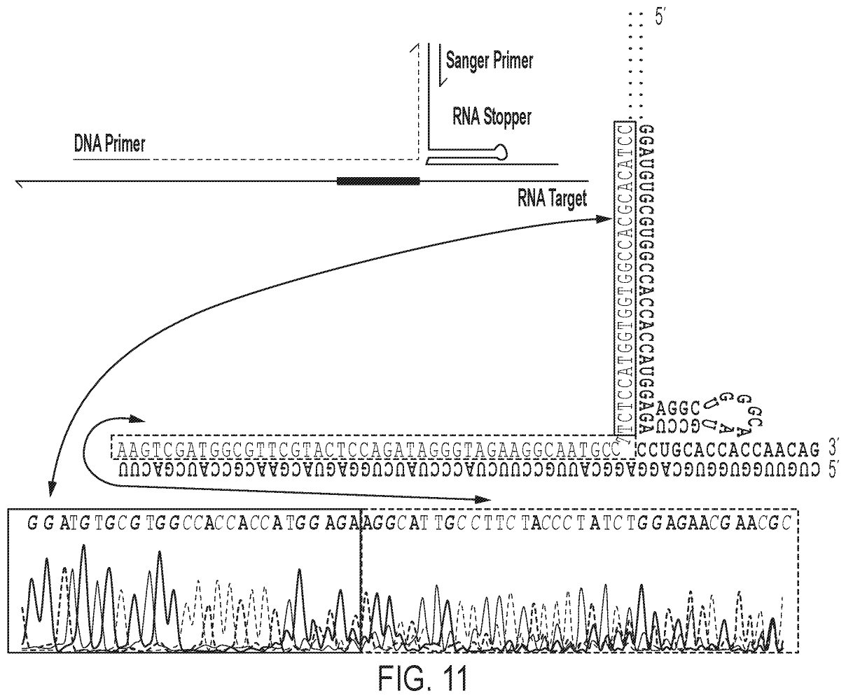

FIG. 11 shows the use of the DNA Primer, RNA Stopper, and the RNA Target to generate an Amplicon in reverse transcription. The three oligonucleotides were first annealed in 2×PBS buffer with 160 units of RNase inhibitor, then the annealed product was mixed with dNTPs and subjected to the 65° C. for 4 minutes followed by a quick chill on ice. After adding 500 units of Maxima H minus reverse transcriptase and 120 units of the RNase inhibitor, the reaction was put under 50° C. for 35 minutes followed by 85° C. for 5 minutes. The Primer and the Sanger primer were used to amplify the generated chimeric Amplicon to achieve the minimum input amount requirement for Sanger Sequencing. Sequencing result was shown in the bottom of FIG. 11, confirming the sequence of the chimeric Amplicons.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising:
(a) a Primer oligonucleotide, and
(b) a Stopper oligonucleotide, wherein the Stopper comprises from 5′ to 3′:
(i) a First Sequence with a length between 5nt and 200nt,
(ii) a Second Sequence with a length between 3nt and 50nt,
(iii) a Loop Sequence with a length between 3nt and 70nt,
(iv) a Third Sequence with a length between 3nt and 50nt, wherein the Third Sequence is complementary to the Second Sequence, and
(v) a Fourth Sequence with a length between 6nt and 500nt,
wherein the Fourth Sequence is complementary to a Binding Region sequence on a Target nucleic acid,
wherein the Third Sequence is complementary to a Match Region sequence positioned to the 3′ of the Binding Region on the Target nucleic acid, and
wherein a 3′ subsequence of the Primer comprising at least 15 nucleotides is complementary to a Priming Region sequence positioned to the 3′ of the Match Region on the Target nucleic acid.

2. The composition of claim 1, wherein the composition is for forming a chimeric amplicon of a Target nucleic acid by polymerase extension, wherein the Target nucleic acid comprises, from 5′ to 3′, a Binding Region, a Match Region, and a Priming Region.

3. The composition of claim 1 or 2, further comprising the Target nucleic acid.

4. The composition of any one of claims 1-3, wherein the Match Region is positioned immediately to the 3′ of the Binding Region.

5. The composition of any one of claims 1-3, wherein the Match Region is adjacent to the Binding Region.

6. The composition of any one of claims 1-5, further comprising a template-dependent polymerase enzyme.

7. The composition of any one of claims 1-6, further comprising reagents and buffers needed for polymerase function.

8. The composition of any one of claims 1-7, wherein the Primer comprises a 5′ subsequence that is not complementary to a region of the Target nucleic acid positioned 3′ of the Priming Region.

9. The composition of any one of claims 1-7, wherein the Primer comprises a 5′ subsequence that is not complementary to a region of the Target nucleic acid positioned immediately 3′ of the Priming Region.

10. The composition of any one of claims 1-7, wherein the Primer comprises a 5′ subsequence that is not complementary to a region of the Target nucleic acid positioned within a 20-nucleotide region 3′ of the Priming Region.

11. The composition of any one of claims 1-10, wherein the Stopper oligonucleotide further comprises a Fifth Sequence between the Second Sequence and the Loop Sequence, and a Sixth Sequence between the Loop Sequence and the Third Sequence, wherein the Fifth Sequence is complementary to the Sixth Sequence.

12. The composition of any one of claims 1-11, wherein the Stopper oligonucleotide has a subsequence at the 3′ end at least 3 nucleotides long that is not complementary to the Target.

13. The composition of claim 12, wherein the subsequence at the 3′ end forms at least one hairpin structure.

14. The composition of any one of claims 1-13, wherein the Stopper oligonucleotide comprises non-natural nucleotides.

15. The composition of any one of claims 1-14, wherein the Stopper oligonucleotide has a chemical functionalization at the 3′ end that prevents polymerase extension.

16. The composition of claim 15, wherein the chemical functionalization is selected from the group consisting of a 3-carbon spacer, an inverted nucleotide, and a minor groove binder.

17. The composition of any one of claims 1-16, wherein the Primer oligonucleotide is a DNA molecule, the Stopper oligonucleotide is a DNA molecule, the Target is a DNA molecule, and the template-dependent polymerase is a DNA polymerase.

18. The composition of any one of claims 1-16, wherein the Primer oligonucleotide is an RNA molecule, the Stopper oligonucleotide is a DNA molecule, the Target is a DNA molecule, and the template-dependent polymerase is a DNA polymerase.

19. The composition of any one of claims 1-16, wherein the Primer oligonucleotide is a DNA molecule, the Stopper oligonucleotide is an RNA molecule, the Target is an RNA molecule, and the template-dependent polymerase is a reverse transcriptase.

20. The composition of any one of claims 1-16, wherein the Primer oligonucleotide is a DNA molecule, the Stopper oligonucleotide is a DNA molecule, the Target is an RNA molecule, and the template-dependent polymerase is a reverse transcriptase.

21. The composition of any one of claims 1-16, wherein the Primer oligonucleotide is an RNA molecule, the Stopper oligonucleotide is an RNA molecule, the Target is an RNA molecule, and the template-dependent polymerase is a reverse transcriptase.

22. The composition of any one of claims 1-16, wherein the Primer oligonucleotide is an RNA molecule, the Stopper oligonucleotide is an DNA molecule, the Target is an DNA molecule, and the template-dependent polymerase is an RNA polymerase.

23. The composition of any one of claims 17-18, wherein the DNA polymerase is selected from the group consisting of Taq DNA polymerase, Bst DNA Polymerase, or DNA Polymerase I, Hemo Klen Taq, Phusion, Q5, T7 DNA polymerase, and KAPA HiFi.

24. The composition of any one of claims 19-21, wherein the reverse transcriptase is selected from the group consisting of Moloney Murine Leukemia Virus reverse transcriptase and Avian Myeloblastosis Virus reverse transcriptase.

25. The composition of any one of claims 1-24, wherein the template-dependent polymerase enzyme is thermostable.

26. The composition of any one of claims 1-24, wherein the template-dependent polymerase enzyme is not thermostable.

27. The composition of any one of claims 1-26, wherein the Target is a biological DNA or RNA molecule.

28. The composition of any one of claims 1-27, wherein the Target is obtained from a sample of cells, a biofluid, or a tissue.

29. The composition of claim 28, wherein the biofluid is selected from the group consisting of blood, urine, saliva, cerebrospinal fluid, interstitial fluid, and synovial fluid.

30. The composition of claim 28, wherein the tissue is a biopsy tissue or a surgically resected tissue.

31. The composition of any one of claims 1-26, wherein the Target is a complementary DNA molecule generated through the reverse transcription of an RNA sample.

32. The composition of claim 31, wherein the RNA sample is a biological RNA sample.

33. The composition of claim 32, wherein the biological RNA sample is obtained from a human, animal, plant, or environmental specimen.

34. The composition of any one of claims 1-26, wherein the Target is an amplicon DNA molecule generated through a DNA polymerase acting on a single-stranded DNA template.

35. The composition of claim 34, wherein the amplicon DNA molecule is generated through multiple displacement amplification of a single cell DNA molecule.

36. The composition of any one of claims 1-26, wherein the Target is a physically, chemically, or enzymatically generated product of a biological DNA molecule.

37. The composition of claim 36, wherein the Target is the product of a fragmentation process.

38. The composition of claim 37, wherein the fragmentation process is ultrasonication or enzymatic fragmentation.

39. The composition of claim 36, wherein the Target is the product of a bisulfite conversion reaction, an APOBEC (“apolipoprotein B mRNA editing enzyme, catalytic polypeptide-like”) reaction, a TAPS (TET-assisted pyridine borane sequencing) reaction, or other chemical or enzymatic reaction in which cytosine nucleotides are selectively converted to uracils based on methylation status.

40. The composition of any one of claims 1-29, wherein the composition comprises a plurality of Stoppers.

41. The composition of claim 40, wherein each of the plurality of Stoppers comprises the same Fourth Sequence.

42. The composition of claim 41, wherein each of the plurality of Stoppers comprises the same Third Sequence.

43. The composition of claim 41, wherein each of the plurality of Stopper comprises a different Third Sequence.

44. The composition of claim 41, wherein multiple Third Sequences are present among the plurality of Stoppers.

45. The composition of claim 40, wherein each of the plurality of Stoppers comprises a different Fourth Sequence.

46. The composition of claim 45, wherein each of the plurality of Stoppers comprises the same Third Sequence.

47. The composition of claim 45, wherein each of the plurality of Stopper comprises a different Third Sequence.

48. The composition of claim 45, wherein multiple Third Sequences are present among the plurality of Stoppers.

49. The composition of claim 40, wherein multiple Fourth Sequences are present among the plurality of Stoppers.

50. The composition of claim 49, wherein each of the plurality of Stoppers comprises the same Third Sequence.

51. The composition of claim 49, wherein each of the plurality of Stopper comprises a different Third Sequence.

52. The composition of claim 49, wherein multiple Third Sequences are present among the plurality of Stoppers.

53. The composition of any one of claims 40-52, wherein the composition comprises a plurality of Primers.

54. The composition of claim 53, wherein each of the plurality of Primers comprises the same 3′ subsequence.

55. The composition of claim 53, wherein each of the plurality of Primers comprises a different 3′ subsequence.

56. The composition of claim 53, wherein multiple 3′ subsequences are present among the plurality of Primers.

57. The composition of any one of claims 1-39, wherein the composition comprises a plurality of Primers.

58. The composition of claim 57, wherein each of the plurality of Primers comprises the same 3′ subsequence.

59. The composition of claim 57, wherein each of the plurality of Primers comprises a different 3′ subsequence.

60. The composition of claim 57, wherein multiple 3′ subsequences are present among the plurality of Primers.

61. The composition of any one of claims 57-60, wherein the composition comprises a plurality of Stoppers.

62. The composition of claim 61, wherein each of the plurality of Stoppers comprises the same Fourth Sequence.

63. The composition of claim 62, wherein each of the plurality of Stoppers comprises the same Third Sequence.

64. The composition of claim 62, wherein each of the plurality of Stopper comprises a different Third Sequence.

65. The composition of claim 62, wherein multiple Third Sequences are present among the plurality of Stoppers.

66. The composition of claim 61, wherein each of the plurality of Stoppers comprises a different Fourth Sequence.

67. The composition of claim 66, wherein each of the plurality of Stoppers comprises the same Third Sequence.

68. The composition of claim 66, wherein each of the plurality of Stopper comprises a different Third Sequence.

69. The composition of claim 66, wherein multiple Third Sequences are present among the plurality of Stoppers.

70. The composition of claim 61, wherein multiple Fourth Sequences are present among the plurality of Stoppers.

71. The composition of claim 70, wherein each of the plurality of Stoppers comprises the same Third Sequence.

72. The composition of claim 70, wherein each of the plurality of Stopper comprises a different Third Sequence.

73. The composition of claim 70, wherein multiple Third Sequences are present among the plurality of Stoppers.

74. A composition for forming a chimeric amplicon of a Target nucleic acid by polymerase extension, wherein the Target nucleic acid comprises, from 5′ to 3′, a Binding Region, a Match Region, and a Priming Region, the composition comprising a Stopper oligonucleotide, wherein the Stopper comprises from 5′ to 3′:
(a) a First Sequence with a length between 5nt and 200nt,
(b) a Second Sequence with a length between 3nt and 50nt,
(c) a Loop Sequence with a length between 3nt and 70nt,
(d) a Third Sequence with a length between 3nt and 50nt, wherein the Third Sequence is complementary to the Second Sequence, and
(e) a Fourth Sequence with a length between 6nt and 500nt,
wherein the Fourth Sequence is complementary to the Binding Region of the Target nucleic acid, and wherein the Third Sequence is complementary to the Match Region of the Target nucleic acid.

75. The composition of claim 74, further comprising the Target nucleic acid.

76. The composition of any one of claims 74-75, wherein the Match Region is positioned immediately to the 3′ of the Binding Region.

77. The composition of any one of claims 74-75, wherein the Match Region is adjacent to the Binding Region.

78. The composition of any one of claims 74-77, further comprising a Primer oligonucleotide, wherein a 3′ subsequence of the Primer comprising at least 15 nucleotides is complementary to a Priming Region sequence positioned to the 3′ of the Match Region on the Target nucleic acid.

79. The composition of any one of claims 74-77, further comprising a template-dependent polymerase enzyme.

80. The composition of any one of claims 74-77, further comprising reagents and buffers needed for polymerase function.

81. The composition of any one of claims 74-78, wherein the Primer comprises a 5′ subsequence that is not complementary to a region of the Target nucleic acid positioned 3′ of the Priming Region.

82. The composition of any one of claims 74-78, wherein the Primer comprises a 5′ subsequence that is not complementary to a region of the Target nucleic acid positioned immediately 3′ of the Priming Region.

83. The composition of any one of claims 74-78, wherein the Primer comprises a 5′ subsequence that is not complementary to a region of the Target nucleic acid positioned within a 20-nucleotide region 3′ of the Priming Region.

84. The composition of any one of claims 74-83, wherein the Stopper oligonucleotide further comprises a Fifth Sequence between the Second Sequence and the Loop Sequence, and a Sixth Sequence between the Loop Sequence and the Third Sequence, wherein the Fifth Sequence is complementary to the Sixth Sequence.

85. The composition of any one of claims 74-84, wherein the Stopper oligonucleotide has a subsequence at the 3′ end at least 3 nucleotides long that is not complementary to the Target.

86. The composition of claim 85, wherein the subsequence at the 3′ end forms at least one hairpin structure.

87. The composition of any one of claims 74-86, wherein the Stopper oligonucleotide comprises non-natural nucleotides.

88. The composition of any one of claims 74-87, wherein the Stopper oligonucleotide has a chemical functionalization at the 3′ end that prevents polymerase extension.

89. The composition of claim 88, wherein the chemical functionalization is selected from the group consisting of a 3-carbon spacer, an inverted nucleotide, and a minor groove binder.

90. The composition of any one of claims 74-89, wherein the Primer oligonucleotide is a DNA molecule, the Stopper oligonucleotide is a DNA molecule, the Target is a DNA molecule, and the template-dependent polymerase is a DNA polymerase.

91. The composition of any one of claims 74-89, wherein the Primer oligonucleotide is an RNA molecule, the Stopper oligonucleotide is a DNA molecule, the Target is a DNA molecule, and the template-dependent polymerase is a DNA polymerase.

92. The composition of any one of claims 74-89, wherein the Primer oligonucleotide is a DNA molecule, the Stopper oligonucleotide is an RNA molecule, the Target is an RNA molecule, and the template-dependent polymerase is a reverse transcriptase.

93. The composition of any one of claims 74-89, wherein the Primer oligonucleotide is a DNA molecule, the Stopper oligonucleotide is a DNA molecule, the Target is an RNA molecule, and the template-dependent polymerase is a reverse transcriptase.

94. The composition of any one of claims 74-89, wherein the Primer oligonucleotide is an RNA molecule, the Stopper oligonucleotide is an RNA molecule, the Target is an RNA molecule, and the template-dependent polymerase is a reverse transcriptase.

95. The composition of any one of claims 74-89, wherein the Primer oligonucleotide is an RNA molecule, the Stopper oligonucleotide is an DNA molecule, the Target is an DNA molecule, and the template-dependent polymerase is an RNA polymerase.

96. The composition of any one of claims 90-91, wherein the DNA polymerase is selected from the group consisting of Taq DNA polymerase, Bst DNA Polymerase, or DNA Polymerase I, Hemo Klen Taq, Phusion, Q5, T7 DNA polymerase, and KAPA HiFi.

97. The composition of any one of claims 92-94, wherein the reverse transcriptase is selected from the group consisting of Moloney Murine Leukemia Virus reverse transcriptase and Avian Myeloblastosis Virus reverse transcriptase.

98. The composition of any one of claims 74-97, wherein the template-dependent polymerase enzyme is thermostable.

99. The composition of any one of claims 74-97, wherein the template-dependent polymerase enzyme is not thermostable.

100. The composition of any one of claims 74-99, wherein the Target is a biological DNA or RNA molecule.

101. The composition of any one of claims 74-100, wherein the Target is obtained from a sample of cells, a biofluid, or a tissue.

102. The composition of claim 101, wherein the biofluid is selected from the group consisting of blood, urine, saliva, cerebrospinal fluid, interstitial fluid, and synovial fluid.

103. The composition of claim 101, wherein the tissue is a biopsy tissue or a surgically resected tissue.

104. The composition of any one of claims 74-99, wherein the Target is a complementary DNA molecule generated through the reverse transcription of an RNA sample.

105. The composition of claim 104, wherein the RNA sample is a biological RNA sample.

106. The composition of claim 105, wherein the biological RNA sample is obtained from a human, animal, plant, or environmental specimen.

107. The composition of any one of claims 74-99, wherein the Target is an amplicon DNA molecule generated through a DNA polymerase acting on a single-stranded DNA template.

108. The composition of claim 107, wherein the amplicon DNA molecule is generated through multiple displacement amplification of a single cell DNA molecule.

109. The composition of any one of claims 74-99, wherein the Target is a physically, chemically, or enzymatically generated product of a biological DNA molecule.

110. The composition of claim 109, wherein the Target is the product of a fragmentation process.

111. The composition of claim 110, wherein the fragmentation process is ultrasonication or enzymatic fragmentation.

112. The composition of claim 109, wherein the Target is the product of a bisulfite conversion reaction, an APOBEC (“apolipoprotein B mRNA editing enzyme, catalytic polypeptide-like”) reaction, a TAPS (TET-assisted pyridine borane sequencing) reaction, or other chemical or enzymatic reaction in which cytosine nucleotides are selectively converted to uracils based on methylation status.

113. The composition of any one of claims 74-107, wherein the composition comprises a plurality of Stoppers.

114. The composition of claim 113, wherein each of the plurality of Stoppers comprises the same Fourth Sequence.

115. The composition of claim 114, wherein each of the plurality of Stoppers comprises the same Third Sequence.

116. The composition of claim 114, wherein each of the plurality of Stopper comprises a different Third Sequence.

117. The composition of claim 114, wherein multiple Third Sequences are present among the plurality of Stoppers.

118. The composition of claim 113, wherein each of the plurality of Stoppers comprises a different Fourth Sequence.

119. The composition of claim 118, wherein each of the plurality of Stoppers comprises the same Third Sequence.

120. The composition of claim 118, wherein each of the plurality of Stopper comprises a different Third Sequence.

121. The composition of claim 118, wherein multiple Third Sequences are present among the plurality of Stoppers.

122. The composition of claim 113, wherein multiple Fourth Sequences are present among the plurality of Stoppers.

123. The composition of claim 122, wherein each of the plurality of Stoppers comprises the same Third Sequence.

124. The composition of claim 122, wherein each of the plurality of Stopper comprises a different Third Sequence.

125. The composition of claim 122, wherein multiple Third Sequences are present among the plurality of Stoppers.

126. The composition of any one of claims 113-125, wherein the composition comprises a plurality of Primers.

127. The composition of claim 126, wherein each of the plurality of Primers comprises the same 3′ subsequence.

128. The composition of claim 126, wherein each of the plurality of Primers comprises a different 3′ subsequence.

129. The composition of claim 126, wherein multiple 3′ subsequences are present among the plurality of Primers.

130. The composition of any one of claims 74-107, wherein the composition comprises a plurality of Primers.

131. The composition of claim 130, wherein each of the plurality of Primers comprises the same 3′ subsequence.

132. The composition of claim 130, wherein each of the plurality of Primers comprises a different 3′ subsequence.

133. The composition of claim 130, wherein multiple 3′ subsequences are present among the plurality of Primers.

134. The composition of any one of claims 130-133, wherein the composition comprises a plurality of Stoppers.

135. The composition of claim 134, wherein each of the plurality of Stoppers comprises the same Fourth Sequence.

136. The composition of claim 135, wherein each of the plurality of Stoppers comprises the same Third Sequence.

137. The composition of claim 135, wherein each of the plurality of Stopper comprises a different Third Sequence.

138. The composition of claim 135, wherein multiple Third Sequences are present among the plurality of Stoppers.

139. The composition of claim 134, wherein each of the plurality of Stoppers comprises a different Fourth Sequence.

140. The composition of claim 139, wherein each of the plurality of Stoppers comprises the same Third Sequence.

141. The composition of claim 139, wherein each of the plurality of Stopper comprises a different Third Sequence.

142. The composition of claim 139, wherein multiple Third Sequences are present among the plurality of Stoppers.

143. The composition of claim 134, wherein multiple Fourth Sequences are present among the plurality of Stoppers.

144. The composition of claim 143, wherein each of the plurality of Stoppers comprises the same Third Sequence.

145. The composition of claim 143, wherein each of the plurality of Stopper comprises a different Third Sequence.

146. The composition of claim 143, wherein multiple Third Sequences are present among the plurality of Stoppers.

147. A method for generating a chimeric Amplicon comprising, from 5′ to 3′, a Primer Sequence, a Match-Complement Sequence, and a First-Complement Sequence, the method comprising:
(a) mixing a Sample comprising a Target molecule comprising, from 5′ to 3′, a Binding Region, a Match Region, and a Priming Region with:
(i) a template-dependent polymerase,
(ii) a Primer oligonucleotide, wherein a 3′ subsequence of the Primer comprising at least 15 nucleotides is complementary to a Priming Region of the Target, and
(iii) a Stopper oligonucleotide, wherein the Stopper comprises from 5′ to 3′
a First Sequence with a length between 5nt and 200nt,
a Second Sequence with a length between 3nt and 50nt,
a Loop Sequence with a length between 3nt and 70nt, and
a Third Sequence with a length between 3nt and 50nt, wherein the Third Sequence is complementary to the Second Sequence and the Match Region of the Target, and
a Fourth Sequence with a length between 6nt and 500nt, wherein the Fourth Sequence is complementary to the Binding Region of the Target, and
(b) incubating the mixture at a temperature conducive to polymerase activity, wherein the Primer Sequence is homologous to the sequence of the Primer oligonucleotide, the Match-Complement Sequence is complementary to the Match Region of the Target, and the First-Complement Sequence is complementary to the First Sequence of the Stopper oligonucleotide.

148. The method of claim 147, wherein step (a) further comprises mixing the Sample with reagents and buffers needed for polymerase function.

149. The method of claim 147 or 148, wherein step (a) comprises mixing the sample with a composition according to any one of claims 1-146.

150. The method of any one of claims 147-149, wherein the Amplicon further comprises an Insert Sequence between the Primer Sequence and the Match-Complement Sequence.

151. The method of any one of claims 147-150, wherein the incubation occurs at a temperature between 10° C. and 74° C. for between 1 second and 20 hours.

152. The method of any one of claims 147-150, wherein the incubation comprises thermal cycling alternating between a temperature higher than 78° C. for between 1 second and 30 minutes and a temperature not higher than 75° C. for between 1 second and 20 hours.

153. The method of any one of claims 147-152, wherein the method further comprises at least 6 additional thermal cycles.

154. The method of any one of claims 147-153, wherein step (a) further comprises mixing the Sample with a fluorophore-functionalized DNA probe, optionally wherein the probe is a Taqman probe or a molecular beacon.

155. The method of any one of claims 147-153, wherein step (a) further comprises mixing the Sample with a DNA intercalating dye, optionally wherein the dye comprises SybrGreen, EvaGreen, or Syto dyes.

156. A method for generating a chimeric Amplicon comprising, from 5′ to 3′, a Primer Sequence, a Match-Complement Sequence, and a First-Complement Sequence, the method comprising:
(a) mixing a Sample comprising a Target molecule comprising, from 5′ to 3′, a Binding Region, a Match Region, and a Priming Region with:
(i) a Primer oligonucleotide, wherein a 3′ subsequence of the Primer comprising at least 15 nucleotides is complementary to a Priming Region of the Target, and
(ii) a Stopper oligonucleotide, wherein the Stopper comprises from 5′ to 3′
a First Sequence with a length between 5nt and 200nt,
a Second Sequence with a length between 3nt and 50nt,
a Loop Sequence with a length between 3nt and 70nt, and
a Third Sequence with a length between 3nt and 50nt, wherein the Third Sequence is complementary to the Second Sequence and the Match Region of the Target, and
a Fourth Sequence with a length between 6nt and 500nt, wherein the Fourth Sequence is complementary to the Binding Region of the Target, and
(iii) an annealing buffer;
(b) thermal annealing the mixture;
(c) adding a template-dependent polymerase, reagents, and buffers needed for enzymatic function; and
(d) incubating the mixture at a temperature conducive to polymerase activity, wherein the Primer Sequence is homologous to the sequence of the Primer oligonucleotide, the Match-Complement Sequence is complementary to the Match Region of the Target, and the First-Complement Sequence is complementary to the First Sequence of the Stopper oligonucleotide.

157. The method of claim 156, wherein step (a) comprises mixing the sample with a composition according to any one of claims 1-146.

158. The method of claim 156 or 157, wherein step (b) comprises a thermocycling program of cooling from a temperature not lower than 78° C. to a temperature not higher than 25° C.

159. The method of claim 158, wherein the thermocycling program comprises steps that cools from 78° C. to 28° C., wherein the solution is held at each 5° C. temperature window for at least 5 minutes.

160. The method of any one of claims 156-159, wherein step (b) comprises incubating the mixture for between 10 minutes to 24 hours.

161. The method of claim 160, wherein step (b) comprises incubating the mixture at room temperature for between 10 minutes to 24 hours.

162. The method of any one of claims 156-161, wherein the Amplicon further comprises an Insert Sequence between the Primer Sequence and the Match-Complement Sequence.

163. The method of any one of claims 156-162, wherein the incubation occurs at a temperature between 10° C. and 74° C. for between 1 second and 20 hours.

164. The method of any one of claims 156-163, wherein the incubation comprises thermal cycling alternating between a temperature higher than 78° C. for between 1 second and 30 minutes and a temperature not higher than 75° C. for between 1 second and 20 hours.

165. The method of any one of claims 156-164, wherein the method further comprises at least 6 additional thermal cycles.

166. The method of any one of claims 156-165, wherein step (a) or step (c) further comprises mixing the Sample with a fluorophore-functionalized DNA probe, optionally wherein the probe is a Taqman probe or a molecular beacon.

167. The method of any one of claims 156-165, wherein step (a) or step (c) further comprises mixing the Sample with a DNA intercalating dye, optionally wherein the dye comprises SybrGreen, EvaGreen, or Syto dyes.
